(19) United States
(12) Patent Application Publication
(10) Pub. No.: US 2023/0310140 A1
(43) Pub. Date: Oct. 5, 2023

(54) A SCAFFOLD FOR A TUBE

(71) Applicant: Ernest LAU, Belfast, County Antrim (GB)

(72) Inventor: Ernest LAU, Belfast, County Antrim (GB)

(21) Appl. No.: 17/800,626

(22) PCT Filed: Feb. 18, 2021
(86) PCT No.: PCT/EP2021/054081

§ 371 Date: Aug. 18, 2022

(30) Foreign Application Priority Data
Feb. 18, 2020 (GB) 2002220.8

(51) Int. Cl. A61F 2/07; A61F 2/844

(52) U.S. Cl. CPC A61F 2/07; A61F 2/844; A61F 2230/0067; A61F 2230/0069

(57) ABSTRACT

A scaffold for a tube, the scaffold having a membrane and a pair of splines integrally formed with or embedded in the membrane. The splines being spaced apart from one another with the membrane spanning therebetween and with the membrane further having a pair of grooves disposed between the splines adapted to receive the splines when the membrane is folded over on itself. The scaffold may be applied internally or externally to a tube, including tubular biological structures (e.g.. arteries) to provide support thereto, and in this sense, it may be used as a stent. The scaffold is more easily deployed and retrieved than known stents,

The present invention relates to a scaffold. In particular, the present invention relates to a scaffold for a tube.

Scaffolds are implanted into tubular biological structures (an artery, a vein, the hepatic duct, the cystic duct, the bile duct, the pancreatic duct, the urethra, the ureter, the oesophagus, the gastric outlet, the duodenum, the colon, the trachea, a bronchus, etc.) to keep the narrowed (“stenosed”) central channel (“lumen”) open and/or prevent leakage through the breached (perforated, ruptured, “dissected”) wall. When used for such purposes, the scaffolds are generally referred to as stents or stent-grafts.

Known stents comprise a scaffold consisting of “struts” which may be coated by polymers or other molecules. The scaffold is generally made of metals or alloys, but may also be made of polymers or composites. The polymer coating (which may comprise several layers) can be passive (i.e. only separating the struts from the biological tissues) or active (i.e. releasing or “eluting” drugs), and durable or biodegradable. Apart from imperfections or defects from the manufacturing processes, microscopic pits may also be intentionally created on the strut surface (usually on the side away from the lumen and towards the wall of the tubular biological structure - “abluminal”) to form reservoirs for drugs. The openings in the scaffold/ between the struts can be spanned by a membrane or fabric to prevent leakage from the lumen (if the tubular structure’s wall has been breached) or encroachment of the lumen from the surrounding environment.

Stents are either self-expanding or expanded with an actuation mechanism (most commonly an inflatable balloon in current clinical practice). A self-expanding stent has a preferred shape due to the intrinsic elasticity of its members and will be able to continue to withstand the constricting influences from elastic recoil of the surrounding biological tissues after deployment. A balloon-expandable stent requires the permanent deformation of some members (dedicated stress concentration points) to achieve the final deployed shape. For a balloon-expandable stent, once the balloon has been deflated, the deployed stent must withstand the constricting influences of the surrounding environment and may be “crushed” or recoil under its intrinsic elasticity to a smaller diameter. To counteract this, balloon expandable stents often need to be expanded to a larger than target diameter during deployment, but over-expansion can cause dissection/ tear in the wall of the tubular biological structure.

Unless a stent is biodegradable, its persistence in a tubular biological structure creates problems: risk of migration, risk of fracture, difficulty/ impossibility of stent retrieval post implantation, and physical bulk (which may cause obstruction to the lumen of the tubular biological structure in which it is implanted). These shortcomings spurred the development of bioabsorbable stents, but their clinical performance has not matched that of the proven metal drug-eluting stents.

Elution (sustained localised gradual release) of anti-proliferative drugs was incorporated into stent design to reduce new overgrowth of the inner lining (“neo-intimal hyperplasia”), which was the main mechanism of in-stent re-stenosis in bare metal stents used in arteries (mainly coronary arteries in clinical practice). However, drug elution creates its own problems, some of which are related to the polymer coatings applied to stent struts in order to hold the drugs.

Anti-proliferative drugs delay arterial wall healing (“endothelialisation” i.e. covering of the stent struts by the normal biological lining of the artery), which in turn can lead to late stent thrombosis (and potentially another heart attack or even death). Incomplete stent endothelialisation is more likely when stent struts are not in direct physical contact with the arterial wall (i.e. stent mal-apposition).

Anti-proliferative drugs (mainly sirolimus) can also lead to “evaginations” (i.e. outward budding of blind sacs from the artenal lumen), especially when the vessel wall is tom (“dissected”) or protrudes between the stent struts into the lumen (“prolapse”) at implantation. These blind sacs are associated with a higher risk of stent mal-apposition, incomplete endothelialisation and stent thrombosis. (Biodegradable stents will inevitably develop strut fracture as the scaffold base material gradually degrades as intended over time, and that can cause stent mal-apposition and evaginations).

Polymer coatings were incorporated into stent design primarily to enable elution. However, these polymer coatings create their own problems. The polymer coatings on stent struts can crack/ fracture, delaminate and form webs and ridges during either manufacture or stent deployment (especially likely if the blood vessel wall is highly calcified). These surface defects cause uneven drug distribution on the stent surface: excessive drug elution may delay endothelialisation: inadequate drug elution may result in neo-intimal hyperplasia. Furthermore, fragments of the polymer coatings can break off as “micro-plastics” and shed into the lumen of the tubular biological structure. If the tubular biological structure is a blood vessel, the polymer fragments are “micro-emboli” that will be washed downstream by blood flow until they are wedged into capillaries too small to allow their passage, effectively blocking them off from the circulation. Micro-embolism of these polymer fragments, together with biological debris released from disruption of atherosclerotic plaques during stretching of the artery, may cause (wholly or partly) the “no-reflow” phenomenon (i.e. no distal blood flow into a previously patent distal artery segment after the patency of the previously narrowed/ occluded proximal segment has been restored by stenting).

The polymers in the coatings, whether they are durable or biodegradable, can induce inflammatory cell and platelet aggregation, which in tum can cause stent thrombosis (blood clotting) and re-stenosis, especially if the surface of the polymer coating has defects.

Stent thrombosis may occur early (0 hours to 30 days post implantation), late (> 30 days to 1-year post implantation) and very late (> 1 year post implantation). Stent thrombosis can acutely occlude a blood vessel, depriving the organ supplied by it of oxygen and other vital nutrients (“ischaemia”). Ischaemia, if prolonged, may lead to irrevocable damage or even death of the entire organ. Thrombosis of a stent in the coronary arteries supplying the heart is associated with a 50 - 70% chance of a heart attack and a 20 - 40 % of sudden death. Stent thrombosis can occur with both bare metal and drug-eluting stents.

The ends of a tubular biological object (the “ostia”) may be slanted with respect to its longitudinal axis if it branches off another tubular biological object or cavity. If an ostium of the tubular object is narrowed and a cylindrical stent is placed inside it, the end of the stent cannot be flush with the ostium: either part of the wall of the tubular object is not covered/ protected by the stent, or a short length of the stent protrudes beyond the ostium (potentially causing obstruction, trapping of luminal contents or inducing thrombosis).

For the coronary arteries, “bifurcation” stenosis involving both the main vessel and a side branch are frequently encountered in clinical practice. Many technologies and techniques have been specifically developed to tackle bifurcation lesions, but they still leave either incomplete vessel wall coverage (“provisional T stenting”) or redundant stent materials protruding beyond the side branch ostium into the lumen of the main vessel (“T stenting and small protrusion” or TAP. “culotte”, mini-crush).

Infection is yet a further problem in known stents. Stents, covered stents and stent-grafts are foreign bodies inside the human or animal body and can be become colonised by bacteria. Once infection has taken hold, biofilms form and bacterial infection becomes very difficult if not impossible to eradicate, Infection of stents, covered stents and stent-grafts can be a persistent and recurrent source of bacteria or related toxins in the blood stream (“septicaemia”), resulting in failure of the scaffold and necessitating its removal from the human or animal body. Infected stents or other biological scaffolds can be very difficult or even impossible to remove through minimally invasive surgery.

Covered stents can stop the ingress of materials or ingrowth from the wall of the tubular biological structure into the stent lumen, and also the egress of materials from the lumen of a breached tubular biological structure into its surroundings. In theory, these functionalities would give covered stents many advantages over uncovered stents, but covered stents also have some disadvantages which stop them from being more widely adopted in practice. The membrane or fabric covering a stent inevitably add rigidity and physical bulk (which can be quite substantial); the covering membrane/ fabric can also impede the deformation or relative movements of the stent struts, and; the mechanical factors make a covered stent less deformable, deliverable and capable of conforming to a tortuous anatomical course

Covered stents can stop the ingrowth from the wall of the tubular biological structure into its lumen, but this also make them more prone to migration post implantation. Flared uncovered ends may mitigate against the migration of covered stents, but the ends may be obstructed by tumour overgrowth and injure the object’s wall because they have to be oversized compared to the tubular biological object in order to achieve fixation The fabric or membrane covering a stent may be resistant to attachment by biological molecules and cells, impeding endothelialisation of the stent if it is implanted in a blood vessel and the covered stent may remain capable of inducing blood clot formation (“thrombogenic”) indefinitely as a result.

Covered stent-grafts and stents are used to treat aortic aneurysms or perforated coronary arteries, but the entrance into any side branch will also be covered. This blockage issue is generally resolved by making windows (“fenestration”) in the covering membrane/ fabric. either before implantation outside the patient’s body or during implantation inside the patient’s body. In the case of intra-procedure fenestration, an angioplasty guide wire with a relatively sharp stiff end, a needle or a powered catheter is needed to perforate the covering membrane/ fabric. The window in the covenng membrane/ fabric and the ostium of the “liberated” side branch need to be reinforced with another stent in order to prevent them from collapsing.

The use of tube scaffolds is not limited to stents in clinical medicine. Another specific area that could benefit from the use of tube scaffolds is in the implantation of electric cables (“leads”) for cardiac implantable electronic devices (pacemakers, implantable cardioverter-defibrillators; referred to as CIEDs) or neuro-stimulators. During CIED implantation, leads are typically inserted from the shoulder (“pectoral”) region of the human body. However, for anatomical reasons, deploying the lead tip at certain specific positions in the heart (e.g. the His bundle, across the inter-atrial septum, into the inter-ventricular septum) is more effectively performed from the groin (“femoral”) region, which means the connector pin of the lead needs to be transferred from the groin region outside the body, through the blood vessels and the heart in the body, and then into the shoulder region outside the body. There are currently no dedicated tools for such a lead transfer process. Doctors performing such manoeuvres (e.g., the Jurdham technique) have had to improvise and modify available medical products to fashion their own tools.

It is an object of the present invention to mitigate or obviate the above-mentioned problems regarding scaffolds for tubes. In particular, it is an object of the present invention to mitigate or obviate the problems associated with: stent deployment; stent retrieval; drug eluting stents; stent thrombosis; ostial coverage even if the ostium is slanted; stent migration; infection of stents; covered stents (rigidity and physical bulk, stent migration, stent non-endothelialisation, and side branch access); the femoral pull through technique for CIED implantation, and; the manufacture and deployment of a flexible tubular-shaped electric battery.

According to an aspect of the invention there is provided a scaffold for a tube, the scaffold comprising a membrane and a pair of splines integrally formed with or embedded in the membrane, the splines being spaced apart from one another with the membrane spanning therebetween, the membrane further comprising a pair of grooves disposed between the splines adapted to receive the splines when the membrane is folded over on itself, wherein one groove is engaged with one spline and the other groove engages with the other spline.

It should be noted that “scaffold” and “stent” may be used interchangeably.

Preferably, the scaffold has a flattened configuration and a rolled configuration.

Preferably, the scaffold is transformable between the flattened and rolled configurations.

Ideally, the scaffold can be reversibly, repeatedly and freely transformed between the flattened and rolled configurations.

Ideally, in the rolled configuration the splines are engaged with the grooves. Advantageously, when the splines are engaged with the grooves the scaffold forms a closed tube with open ends or a truncated cone with open ends, and is capable of providing support to another tube, including tubular biological structures.

Preferably, in the rolled configuration the splines and grooves are helical in shape.

Ideally, the grooves are parallel with one another or diverging/converging from one another, and they are straight or curved.

Preferably, the grooves do not overlap one another and are discrete from one another.

In one embodiment, the scaffold is substantially cylindrical in the rolled configuration.

In another embodiment, the scaffold is substantially conical in the rolled configuration.

Ideally, in the rolled configuration, the grooves and splines are overlapping spiral helices.

Ideally, the cone has a narrow diameter end near the apex and a wide diameter end at the base.

Preferably, the grooves and splines diverge from one another in a direction from the narrow diameter end towards the wide diameter end.

Ideally, the amount of membrane between the splines and grooves increases in a direction from the narrow diameter end towards the wide diameter end.

Ideally, in the rolled configuration, the cone is truncated.

Preferably, the cone has a circular base

Ideally, in the rolled configuration, the splines and grooves form intertwining spiral helices with a transverse diameter that decreases in a direction from the base of the cone to the apex.

Ideally, the scaffold forms a right circular cone when in the rolled configuration.

Preferably, the scaffold is configurable as a helix formation.

By “helix formation”, we mean the splines and grooves are substantially helical in shape.

Ideally, the scaffold is telescopic in the rolled configuration such that it can longitudinally expand or retract.

Preferably, the diameter of the scaffold in the rolled configuration is operably adjustable.

Ideally, longitudinally extending the scaffold in the rolled configuration reduces its diameter.

Preferably, the scaffold is configurable as a telescopic cylindrical or conical helix formation.

Ideally, the scaffold is configurable as a telescopic cylindrical or conical helix formation by rolling up a flat scaffold membrane patch.

Ideally, the scaffold self assembles into the rolled configuration.

Preferably, the splines and/or the receiving grooves are formed of shape memory materials that will assume a pre-set spiral shape at predetermined temperature such that the scaffold self assembles into the rolled configuration at a predetermined temperature.

Preferably, the splines and/or the receiving grooves are formed of either shape memory materials that will assume a pre-set helical or conical spiral shape (spontaneously or in response to actuation), or malleable materials that will retain the shape after non-elastic deformation.

Ideally, the receiving grooves match substantially half of the profile of the splines.

Ideally, the longitudinal orientation of the grooves are opposing so that one groove extends out of the membrane on one side of the membrane and the other groove extends out of the membrane on the other side of the membrane.

Preferably, the cross sections of the splines are circular, elliptical, rectangular, triangular or any other regular or irregular geometric shapes.

Preferably, the pair of splines are principal splines and the scaffold comprises one or more auxiliary splines.

Ideally, the auxiliary spline or splines are disposed proximal to one or both longitudinal ends of the scaffold.

Preferably, the auxiliary spline or splines are made of materials having shape memory.

Ideally, the auxiliary spline or splines extend fully or partially between the principal splines

Ideally, the scaffold comprises one or more handles to facilitate deployment and retrieval of the scaffold.

In one embodiment, the one or more handles are formed from auxiliary splines.

Ideally, the one or more handles are formed from a material having shape memory such as nitinol.

ideally. the one or more handles may be deformed and are configured to return to a pre-set shape upon being heated to a predetermined temperature.

Preferably, the one or more handles may be positioned such that entry into the central hollow of the scaffold in the rolled configuration by a retrieval tool. such as an inflatable balloon, is not prevented.

ideally, the one or more handles may be folded away from the longitudinal axis of the scaffold in the rolled configuration

Preferably, upon reaching a pre-set temperature, the one or more handles fold towards the longitudinal axis of the scaffold in the rolled configuration. Advantageously, this narrows a part of the scaffold lumen. In use, a balloon may be inserted into the scaffold and inflated with a liquid that warms the one or more handles to the pre-set temperature, causing the one or more handles to fold towards the central axis of the lumen. The balloon may then be partially deflated and pulled, the handle now creating a blockage to movement of the balloon through the scaffold and enabling retrieval of the scaffold.

In one embodiment, the scaffold has internal handles that do not protrude outside the scaffold membrane. splines and grooves.

In another embodiment, the scaffold has external handles that protrude outside the scaffold membrane, splines and grooves.

In yet another embodiment, the scaffold has both internal and external handles.

In some embodiments, the handles may be detachable.

In one embodiment, the handle is anchorable to a surface, e.g., via sutures or the like.

Ideally, the handle comprises an aperture to receive an anchoring means such as a suture. Advantageously, the scaffold can be applied to a structure such as a lead, and the handle used to anchor the lead to a surface, such as biological tissues.

Ideally, the scaffold membrane may be impregnated with a lubricant such as perfluorocarbons.

In one embodiment, the scaffold comprises an auxiliary spline and an auxiliary groove, the auxiliary groove being positioned to receive the auxiliary spline. Advantageously, a series of such scaffolds can form a continuous surface by the auxiliary spline of one scaffold slotting into the auxiliary spline of an adjacent scaffold.

In one embodiment, wherein the splines are parallel to the grooves, the width-wise distance from the first spline to the first groove is the same width-wise distance as that from the second spline to the second groove.

In another embodiment, wherein the membrane when flattened is trapezoidal in shape and bound by curved lines rather than straight parallel lines, the angular distance from the first spline to the first groove is equal to that of the angular distance from the second spline to the second groove.

Preferably, the splines and the grooves are constructed out of a single material (e.g. a metal, an alloy, a polymer, a copolymer) or a composite of several materials (e.g. a metal alloy, a mixture of polymers, a polymer doped with inorganic compounds, a polymer reinforced with microfibrils of other materials, etc.).

In one embodiment, the scaffold membrane comprises polytetrafluoroethylene, most preferably, expanded polytetrafluoroethylene (ePTFE).

Preferably, the membrane is formed from two or more membrane layers.

Ideally, the membrane comprises two or more layers of ePTFE.

Preferably, the membrane comprises a core layer sandwiched by outer layers.

Preferably, the core layer is more rigid than one or both outer layers.

Preferably, the membrane comprises fluorinated ethylene propylene (FEP).

Ideally, the core layer is formed from fluorinated ethylene propylene (FEP).

In another embodiment, the scaffold membrane comprises a bioabsorbable polymer such as polylactic acid (PLA), poly-L-lactic acid (PLLA), poly-D-lactic acid (PDLA), poly-LD-lactic acid (PDLA), polyglycolic acid (PGA), poly(lactic-co-glycolic acid) co-polymer (PGLA), polycarprolactone (PCL). poly(glycolide-co-caprolactone) co-polymer (PGCL), polydioxanone (PDX), polyorthoesters (POE), or combinations thereof.

In one embodiment, the membrane is formed entirely from a single layer of bioabsorbable polymer.

The principal and/or auxiliary spline-groove joints may be left bare or sealed with an adhesive that may be rigid (e.g. a resin) or elastic (e.g. an elastomer) when set. In this embodiment, the adhesive may be pressure activated. Additionally, or alternatively, the adhesive may comprise two components that, when mixed, begin curing. Advantageously, one component may be disposed on the splines and another on the grooves so that when they are brought into contact the adhesive is activated.

The scaffold may be engineered to release molecules into the surrounding environment and may thereby be drug-eluting. In this embodiment, the membrane can be engineered to release molecules into the surrounding environment.

In one embodiment, the scaffold is a parallelogram with acute and obtuse internal angles (i.e < 90° or > 90°, but ≠ 90° in its flattened configuration, wherein the splines form the longer edges.

Ideally, when transformed from the flattened configuration into the rolled configuration, a spline engages with a groove that is further away and not immediately adjacent. Advantageously, this engagement retains the scaffold in the rolled configuration with overlap of layers of the scaffold membrane in certain sections.

Preferably, in the rolled configuration, the scaffold forms a continuous surface of alternating single-layered, double-layered or multi-layered wall thickness.

Ideally, in the rolled configuration, the splines and grooves form helices whose turns directly stack on one another in the neutral unbent state.

Preferably, when the scaffold in the rolled configuration is bent along its longitudinal axis, the overlap between turns of the scaffold ensures a continuous surface is maintained, even if part of the splines is no longer locked in the receiving grooves.

Ideally, the longitudinal span of the scaffold in the rolled configuration can be increased or decreased.

Ideally, the splines slidably engage with the grooves so that the spline can be slid along a groove when in the rolled configuration.

Preferably, the longitudinal span of the scaffold in the rolled configuration can be increased or decreased by winding up the turns of the splines and grooves into helices of larger or smaller pitches (with corresponding smaller or larger transverse diameters).

Ideally, the scaffold in the rolled configuration may have flush ends, wherein the scaffold terminates in ends defining planes that are orthogonal to the longitudinal axis of the scaffold, or staggered, wherein the ends stagger in the direction of the longitudinal axis of the scaffold.

Alternatively, one longitudinal end may be flush, and the other end staggered.

In one embodiment, the scaffold may have a plurality of rolled configurations, wherein different rolled configurations provide different diameters.

In one embodiment, the scaffold comprises a plurality of pairs of grooves.

Ideally, in one rolled configuration, the splines are locked with one pair of grooves, whereas in another rolled configuration the splines are locked with a different pair of grooves.

Preferably, the diameter of the rolled configuration doubles, halves or alters in any ratio when transforming between the different rolled configurations.

In one embodiment, the scaffold has a first rolled configuration and a second rolled configuration. The first rolled configuration may be half the diameter of the second rolled configuration.

In one embodiment, the splines have a teardrop shaped cross-section and the grooves are correspondingly shaped to receive the splines, with the cross-section of one groove corresponding to the pointed end of the teardrop shaped spline, and the other groove being shaped to correspond to the rounded end of the teardrop shaped spline.

In one embodiment, the scaffold has a spline-groove arrangement wherein the splines project from the surface of the membrane and have spaces to either lateral side of the spline to receive the groove, which envelopes the spline at either lateral side thereof.

In one embodiment, the thickness of the membrane is variable.

Ideally, the thickness of the membrane is greater in the space between the grooves than in the space between either spline and said spline’s nearest groove.

Preferably, the thickness of the membrane is substantially or exactly doubled in the space between the grooves than in the space between either spline and said spline’s nearest groove.

Ideally, in the flattened configuration, a first planar surface of the membrane extends from the first spline to the second groove, and a second planar surface extends from the second spline to the first groove, overlapping in the space between the grooves where the membrane is doubled in thickness.

In one embodiment, the scaffold is fixable to a pin plug for connecting the scaffold to the connector pin of a lead used with a CIED or a neuro-stimulator.

Ideally, the scaffold is joinable to the base of a pin plug.

Preferably, the pin plug comprises a female connecting means for connecting to the connector pin for a lead.

Ideally, the female connecting means comprises a central cylindrical core surrounded by a cylindrical shell, with the space between the core and shell being sized to receive the connector pin of a lead.

Preferably, the central cylindrical core and the cylindrical shell or mounted on a plate Ideally, the pin plug comprises a handle configured to receive a snare or other grasping device. Preferably, the handle has a neck and a wide portion.

In one embodiment, the scaffold is adapted for use in the manufacture of batteries.

Ideally, the scaffold has a pair of receiving groves located adjacent to the splines.

Preferably, the scaffold membrane has a plurality of layers.

Preferably, the scaffold membrane comprises a current conductor strip.

Ideally, the scaffold membrane comprises a cathode.

Preferably, the current conductor strip and the cathode are sandwiched between structural layers.

Ideally, one layer on one side of the cathode and conductor strip is permeable to ions (electrolytes) and solvents whereas one layer on the other side is impermeable to ions (electrolytes) and solvents.

Ideally. the scaffold membrane comprises, in order being arranged from the exterior to interior when in the rolled configuration: one or more layers of ePTFE, a laminating layer of FEP, a thin (cathode) current conductor strip (e.g. made out of aluminium foil), a cathode (e.g. carbon monofluoride, manganese dioxide, generally mixed with other binding materials into a paste), and one of more layers of ePTFE (semi-permeable).

Advantageously, the scaffold can be wrapped around a central anode core (e.g. lithium metal) containing a central (anode) current collector which is also malleable/ flexible (e.g. a copper wire, a silver wire, an aluminium wire, a graphene string, a carbon nanotube construct).

Ideally, the spline-groove joints are sealed with an elastic but impermeable adhesive.

Advantageously, lithium is highly malleable and can be easily be shaped with grooves or indentations to accommodate the bulges of the cathode paste. The semi-permeable luminal ePTFE layers allow the passage of ions (electrolytes) and solvents and can be made to be extremely thin to minimise the internal resistance of the battery. The luminal layers can also be made to be extremely strong against tear (e.g by orienting successive layers of ePTFE so that their fibrils lie orthogonally) to prevent the cathode and the anode coming into direct physical contact (which would generate an internal short circuit of the battery and a runaway electrochemical and thermal reaction). The FEP laminating layer seals up the entire battery (except for connections for the current collectors) and prevents the leakage of its contents (mainly the solvents).

Ideally, the external layer is impregnated with a perfluorocarbon. Advantageously, this renders the entire battery resistant against tissue ingrowth, thrombosis (blood clot formation) and bacterial colonisation. Such a flexible cylindrical high energy density will be very useful for powering CIEDS (e.g. a leadless pacemaker, an implantable “string” subcutaneous defibrillator). However, the same battery will also be useful for powering other non-medical consumer electronic products.

According to a further aspect of the invention there is provided a scaffold for a tube that can be deployed inside the tube to engage with and provide support to said tube, the diameter of the scaffold being operably adjustable and the scaffold further being retrievable by operably reducing the diameter of the scaffold such that it disengages from the tube and can be removed from the tube.

Ideally, the scaffold is configured such that the diameter of the scaffold can be adjusted remotely, using one or more tools to adjust the diameter of the scaffold from a location distal to that of the scaffold.

According to a further aspect of the invention there is provided a method for retrieving a scaffold from a tube, the method comprising the step of inserting an inflatable balloon into the lumen of the scaffold and inserting a heated substance into the balloon to inflate the balloon and heat the scaffold such that the shape of the scaffold is altered by the heat thereby trapping the balloon in the scaffold, then drawing the balloon and the scaffold out of the tube.

According to a further aspect of the invention there is provided a method of extracting a lead from a human or animal body, the lead being enveloped by a scaffold having two nitinol principal splines and a nitinol auxiliary spline, the method comprising the steps of applying an electric current to the splines resulting in the splines heating and assuming their predetermined shape resulting in radial expansion of the scaffold thereby urging the surrounding tissues away from the lead, the method then comprising removing the lead by drawing it out from the scaffold.

Ideally, the method comprising the step of inserting a locking stylet or lead locking device inside the lumen of the lead to provide tensile strength and distal lead tip control.

Preferably, the method composing the step of inserting a sheath around the lead through the channel newly created within the radially expanded scaffold.

Ideally, the method comprising removing the lead with the locking stylet or lead locking device.

Preferably, the method comprising inserting a guide wire through the sheath.

Ideally, the method comprising removing the scaffold by pulling on its proximal end around the guide wire.

According to a further aspect of the invention there is provided a method for inserting a transvenous lead, the method comprising initially applying a pin plug and scaffold arrangement to a lead pin, positioning said arrangement with a ramrod dilator, applying a snare to the handle of the pin plug, and drawing the lead through a sheath via the snare.

According to a further aspect of the invention there is provided a method of applying a conical scaffold at a slanted ostium, the method comprising urging the end of the scaffold flush or near flush with the slanted ostium using an inflatable balloon.

Ideally, the method comprising the step of initially inserting the scaffold applied to a deflated balloon in a non-expanded state into the slanted ostium, using a guide wire.

Preferably, the method comprising inflating the balloon to expand the scaffold.

Ideally, the method comprising removing the balloon and inserting a second shorter balloon via a guide wire into the scaffold at the portion where the scaffold is proximal in the slanted ostium and inflating said balloon.

Preferably, the method comprising pulling the second shorter balloon over the guide wire to draw the proximal scaffold out past the slanted ostium.

Ideally, if there is a wall opposing the slanted ostium, the method comprises inserting a deflated balloon via a guide wire along the opposing wall such that it opposes the scaffold, and inflating the balloon so that it abuts the opposing wall and urges the scaffold to make it flush or near with the slanted ostium.

Alternatively, the urging balloon can be inserted in a guide catheter and inflated so that the guide catheter prevents the balloon from being displaced away from the scaffold when it contacts the scaffold, the scaffold then being urged flush with the slanted ostium.

Ideally, the method comprising removing the balloon.

According to a further aspect of the invention there is provided a method for manufacturing a battery, the method comprising the steps of providing a central anode core (e.g. lithium metal) containing a central (anode) current collector which is also malleable/ flexible (e.g. a copper wire, a silver wire, an aluminium wire, a graphene string, a carbon nanotube construct), and wrapping the central anode core with a scaffold, the scaffold comprising a current conductor strip and a cathode.

According to a further aspect of the invention there is provided a battery, the battery comprising a scaffold for a tube.

Ideally, the scaffold forms an outer layer of the battery.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

The invention will now be described with reference to the accompanying drawings in which:

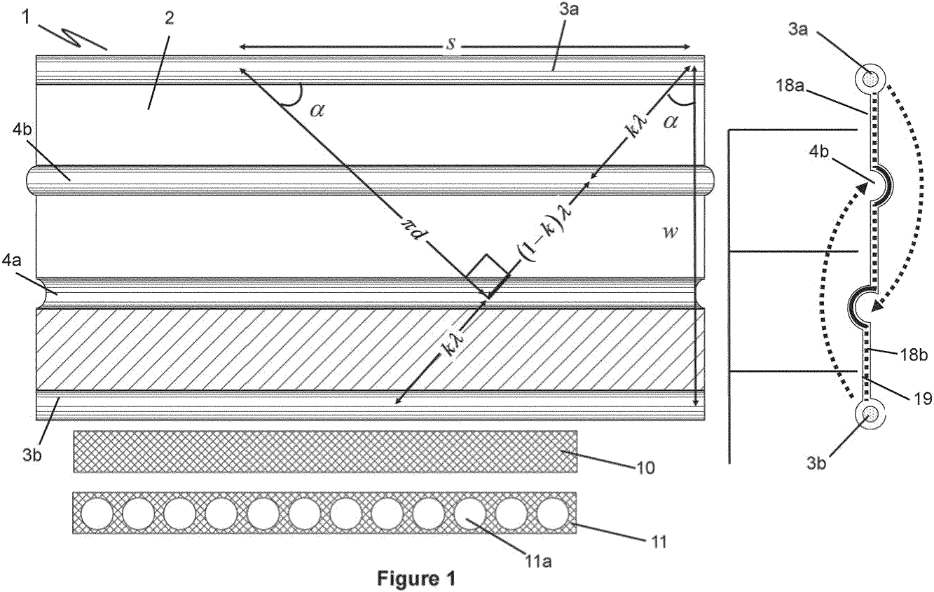

FIG. 1 shows a scaffold for a tube according to the invention, the scaffold being unrolled and flattened.

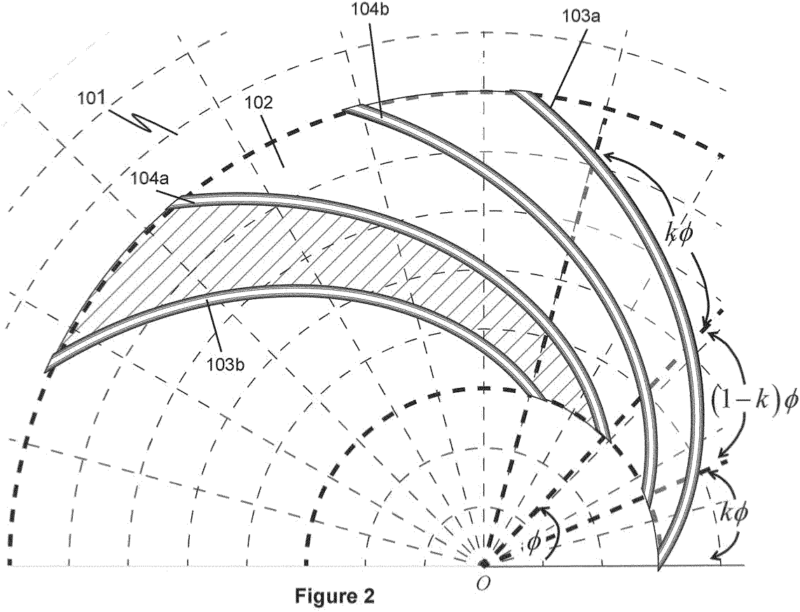

FIG. 2 shows a further embodiment of a scaffold according to the invention in a flattened configuration

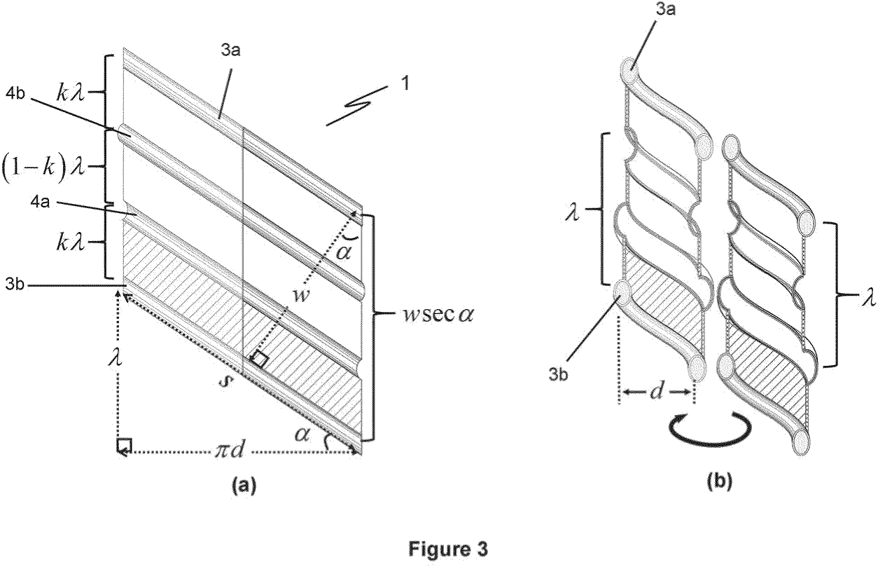

FIG. 3 shows the scaffold of FIG. 1 in (a) flattened, and (b) rolled configurations. The rolled configuration (b) is depicted in cross section.

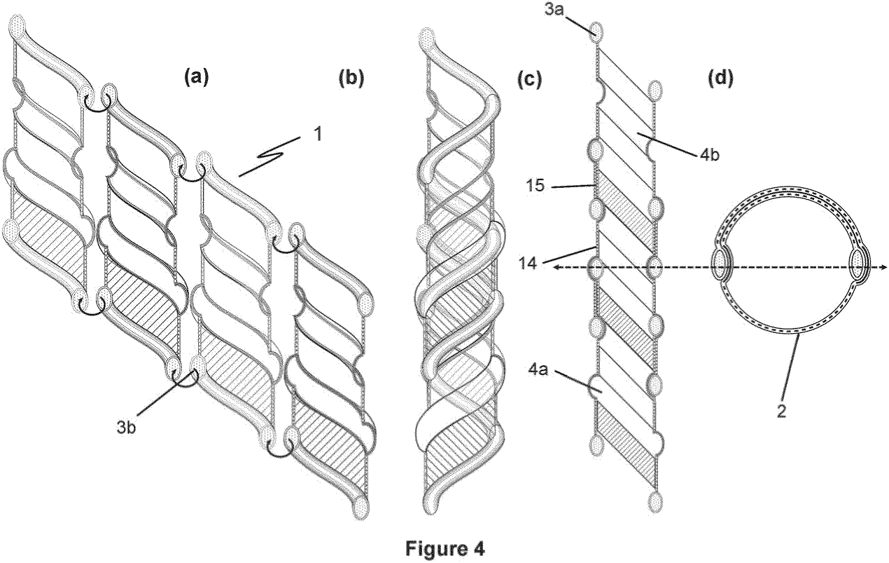

FIG. 4 shows the scaffold of FIG. 1 in the rolled configuration as (a) a series of cross sections, (b) overlapping, layered cross sections, (c) overlapping cross sections, and (d) transverse section.

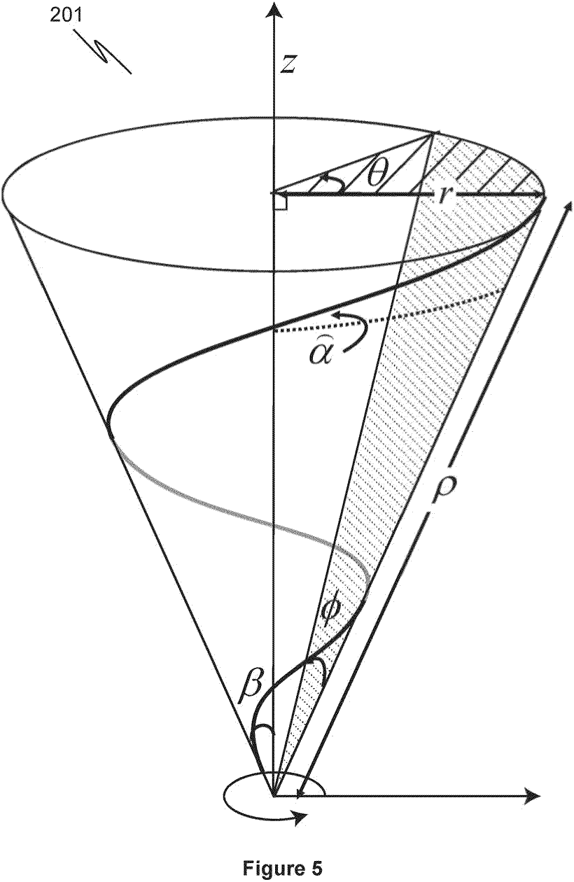

FIG. 5 is a schematic representation of a conical scaffold according to an embodiment of the invention when in the rolled configuration.

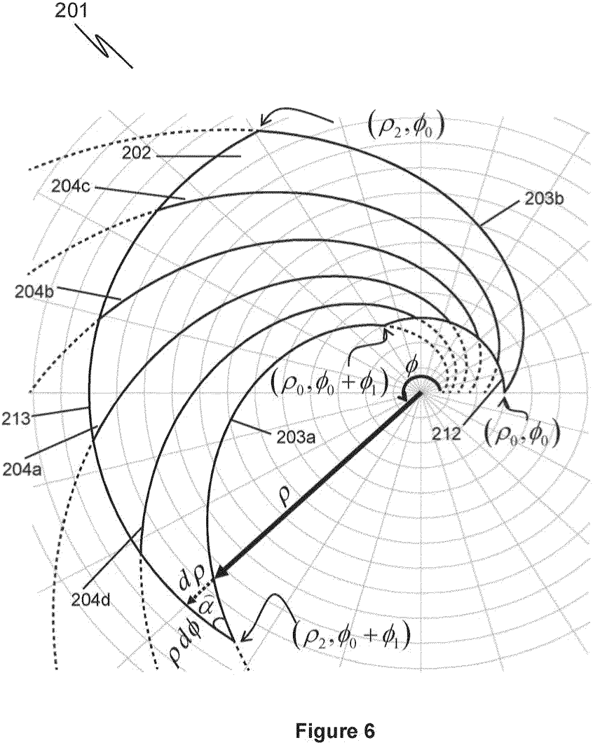

FIG. 6 shows the conical scaffold of FIG. 5 when flattened.

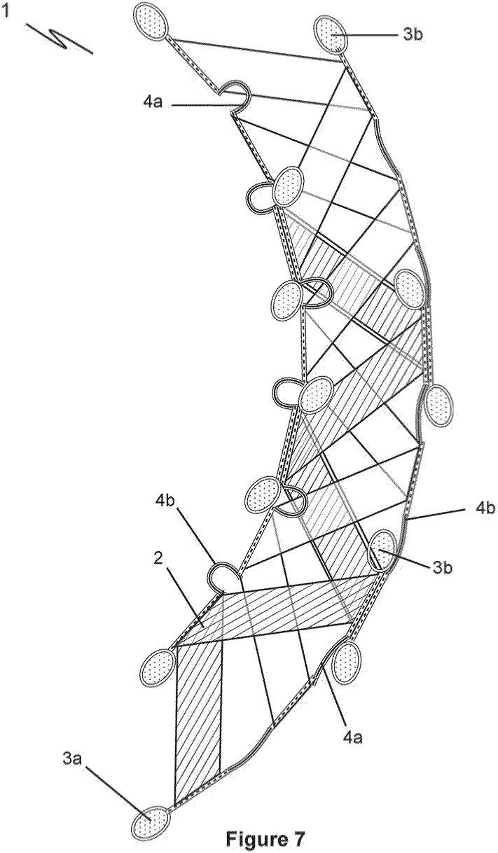

FIG. 7 shows a cross-sectional view of the scaffold of FIG. 1 when in the rolled configuration and when the longitudinal axis is bent.

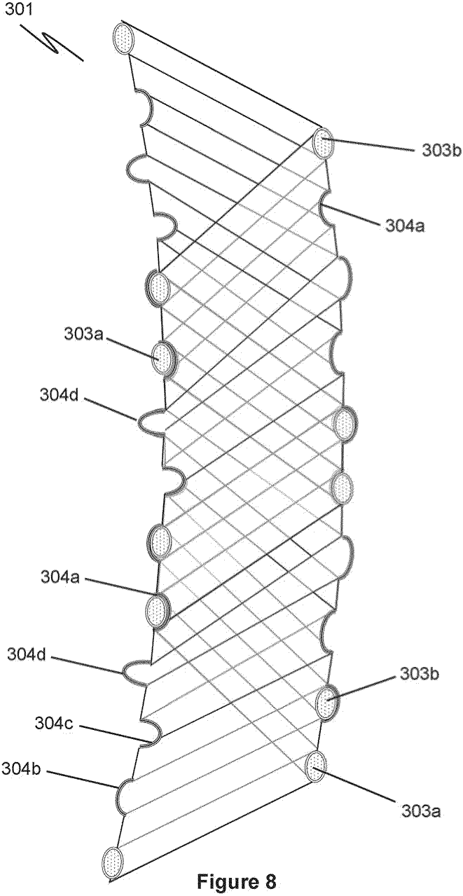

FIG. 8 shows a cross section of a further embodiment of a scaffold according to the invention.

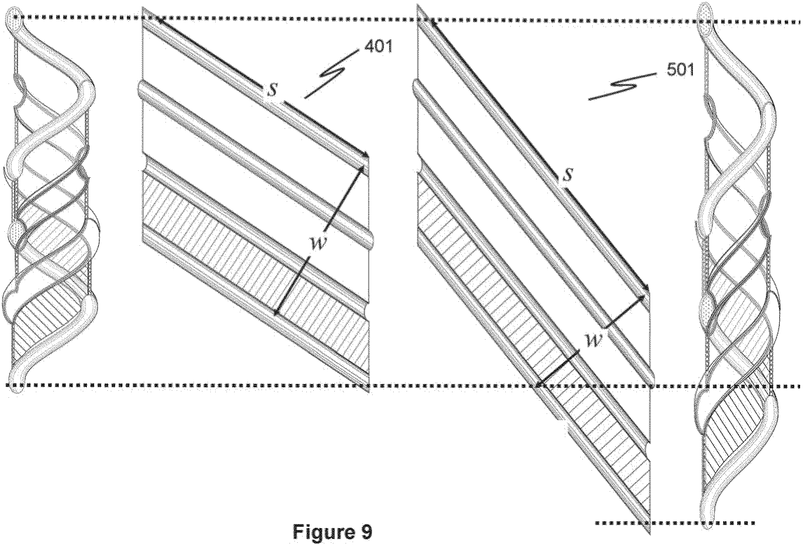

FIG. 9 shows further embodiments of scaffolds according to the invention in flattened and rolled configurations.

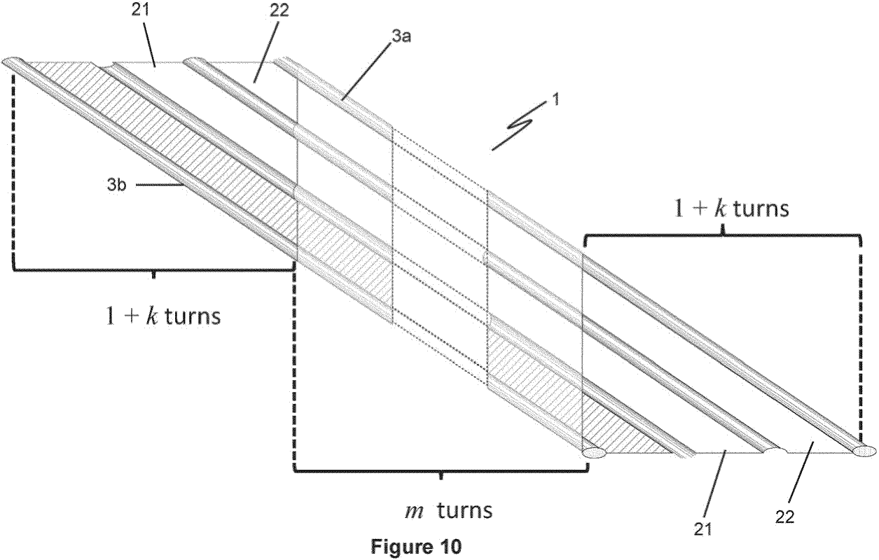

FIG. 10 shows a modification of the first embodiment of a scaffold in the flattened configuration.

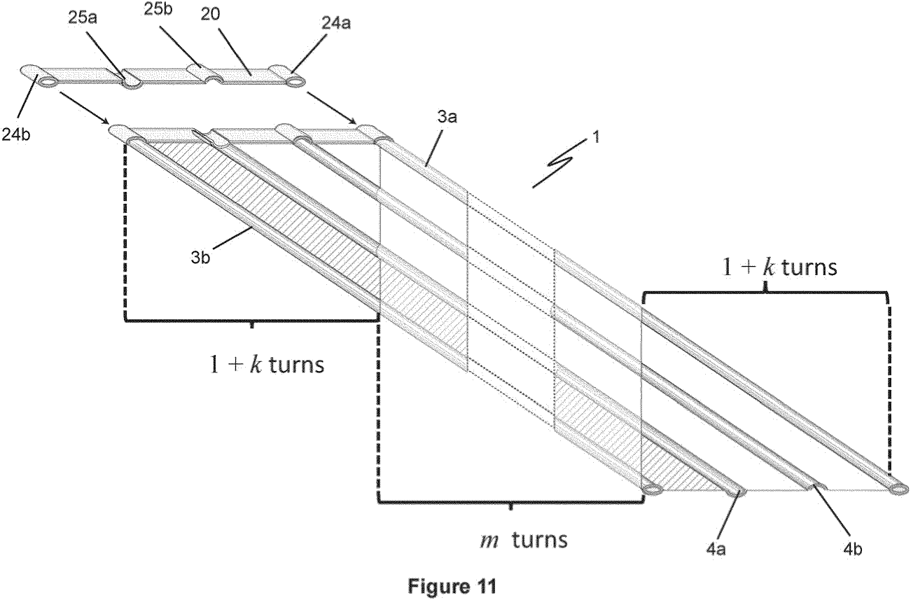

FIG. 11 shows a further modification of the first embodiment of a scaffold in the flattened configuration.

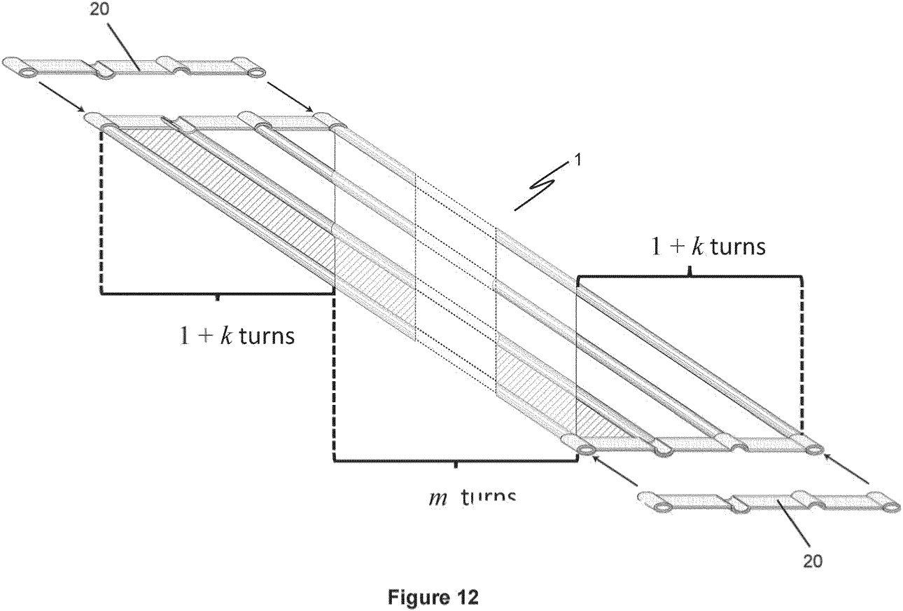

FIG. 12 shows a further modification of the first embodiment of a scaffold in the flattened configuration.

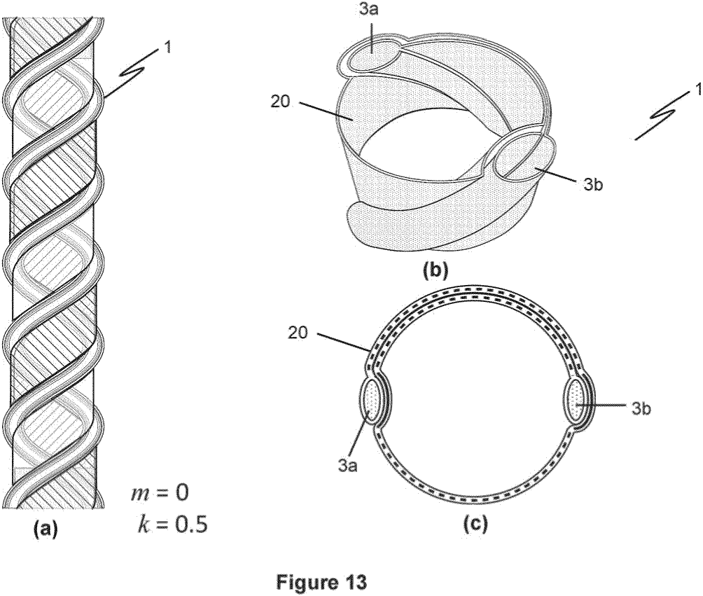

FIG. 13 shows the scaffold of FIG. 12 rolled in (a) front elevation view, (b) end perspective view, and (c) end view.

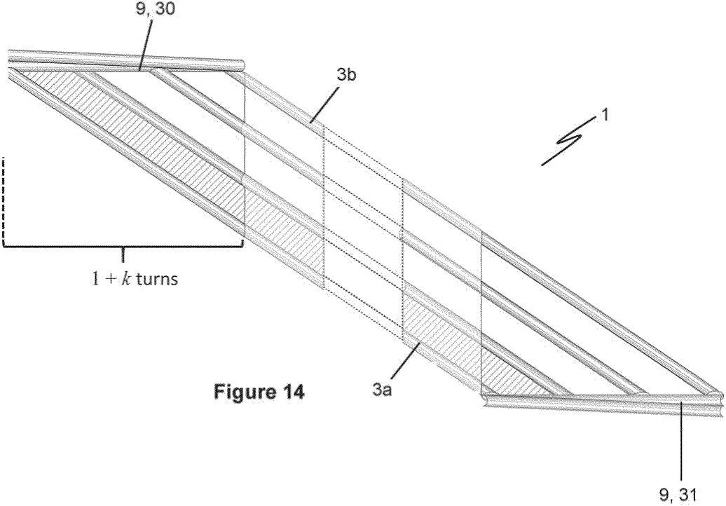

FIG. 14 shows a further modification of the first embodiment of a scaffold in the flattened configuration.

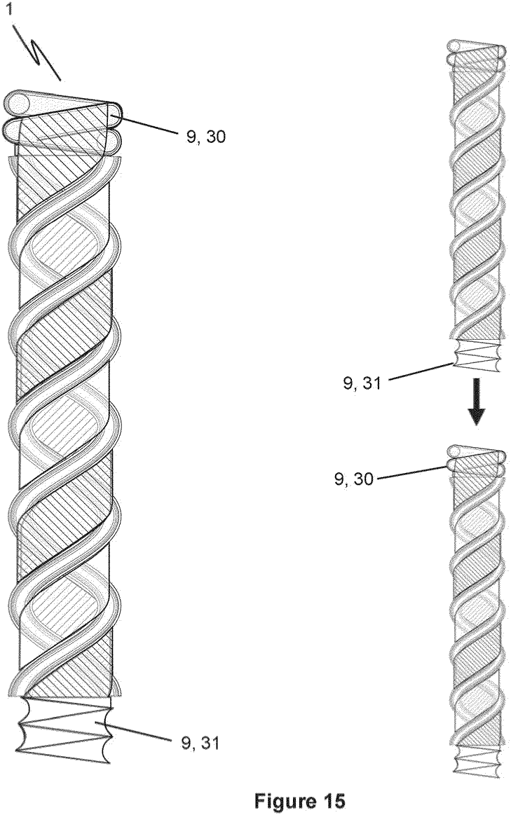

FIG. 15 shows the scaffold of FIG. 14 in the rolled configuration.

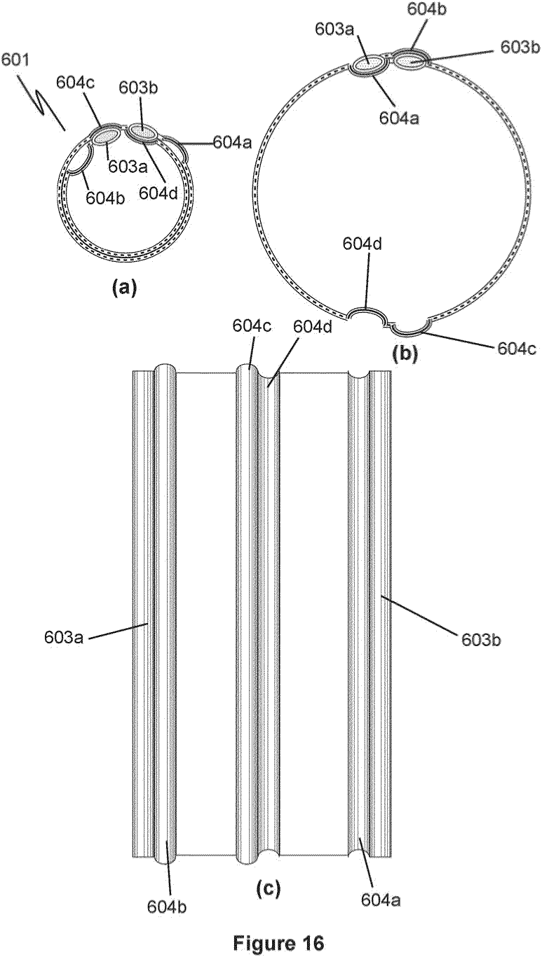

FIG. 16 shows a further embodiment of a scaffold according to the invention in (a) an end view of a first rolled configuration, (b) end view of a second rolled configuration and, (c) flattened configuration.

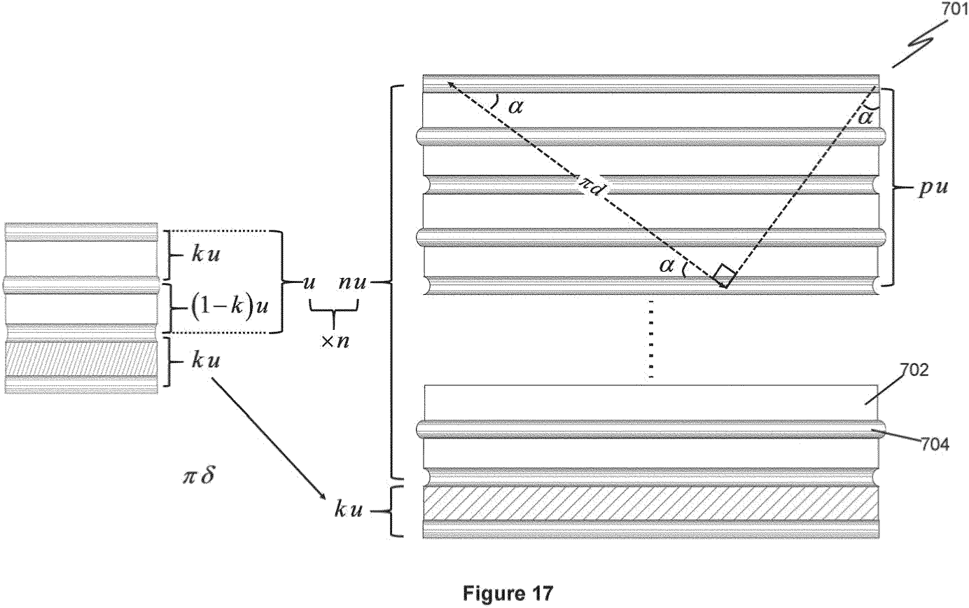

FIG. 17 shows a further embodiment of a scaffold according to the invention in the flattened configuration.

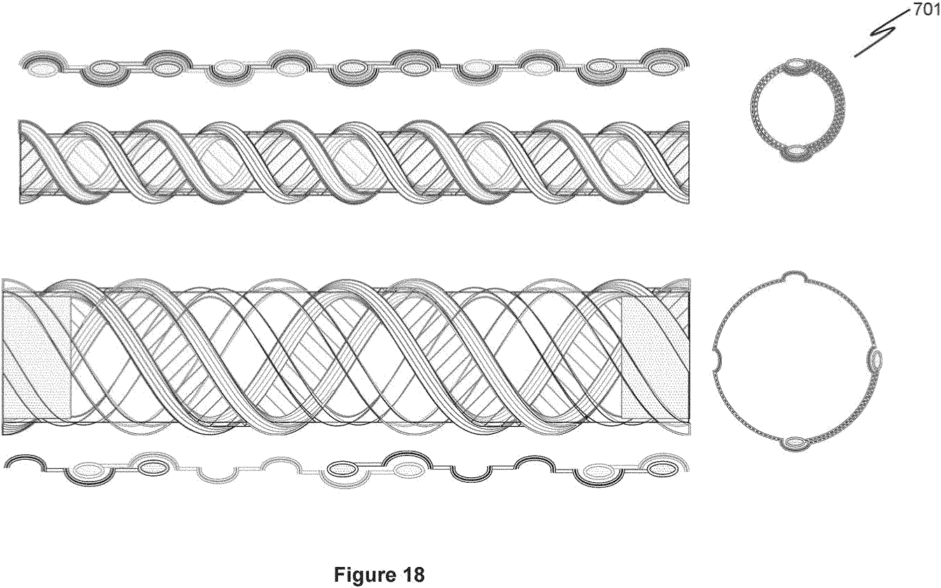

FIG. 18 shows the embodiment of FIG. 17 in the rolled configuration.

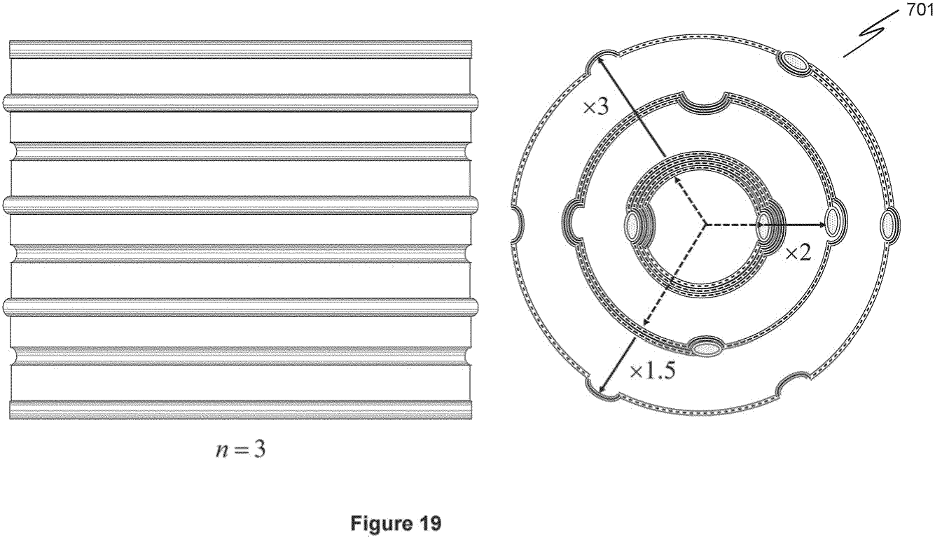

FIG. 19 shows the embodiment of FIG. 17 in flattened and rolled configurations.

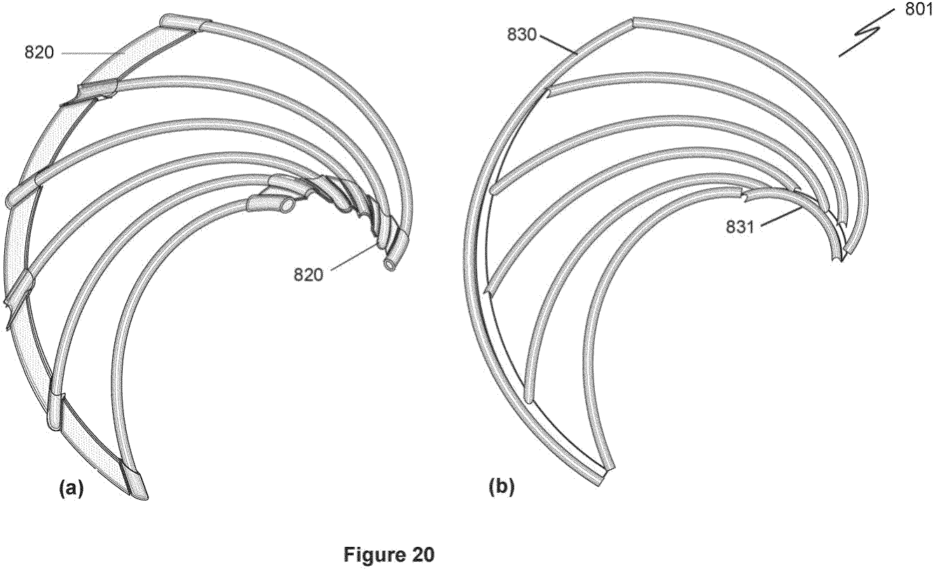

FIG. 20 shows a further embodiment of a scaffold according to the invention in the flattened configuration.

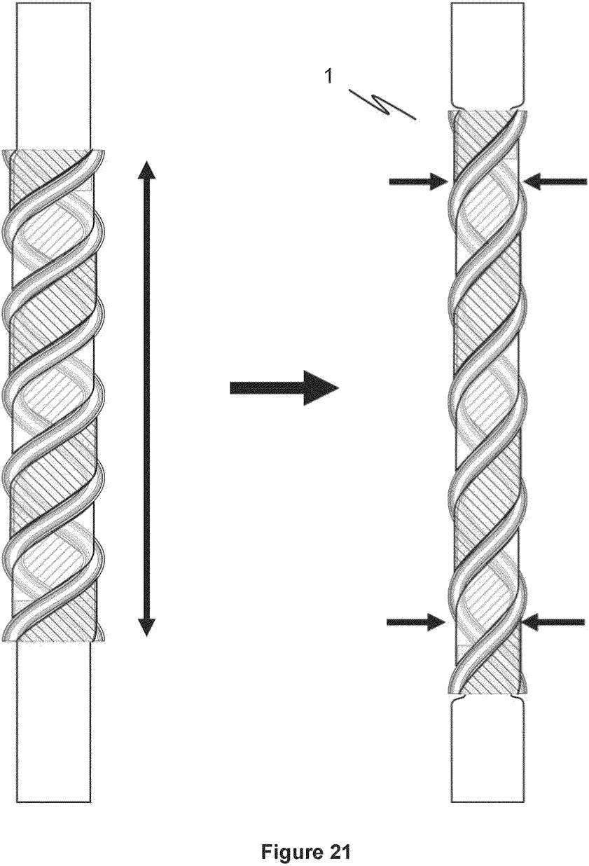

FIG. 21 shows the scaffold of FIG. 1 in use.

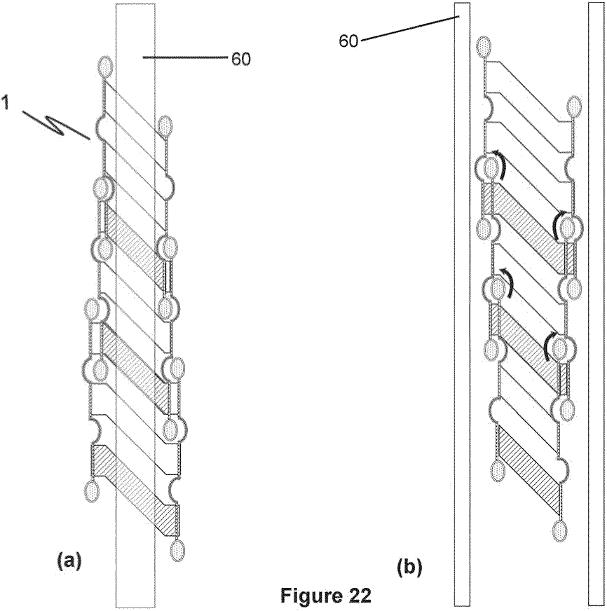

FIG. 22 shows the scaffold of FIG. 1 in use when applied (a) externally on a tubular object and (b) internally.

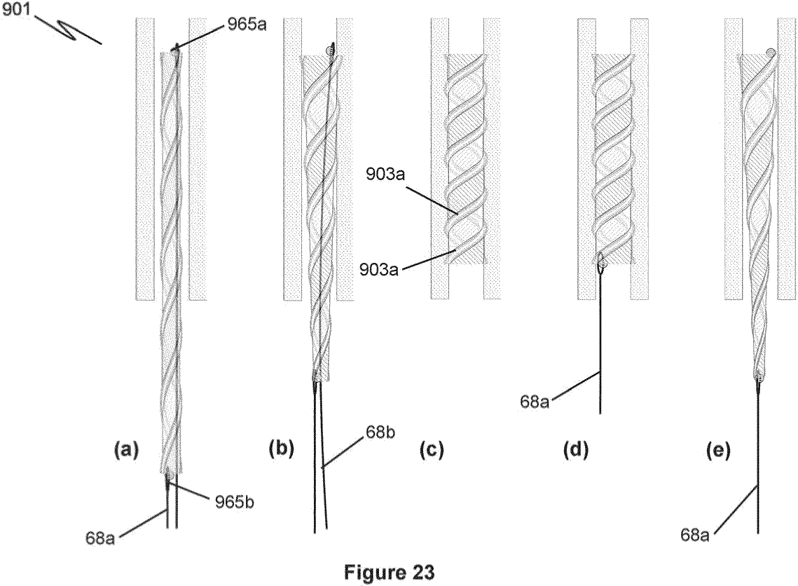

FIG. 23 shows a further embodiment of a scaffold according to the invention, the scaffold is (a) longitudinally extended during deployment via handles, (b) being positioned during deployment, (c) deployed, (d) prior to retrieval, and (e) being retrieved.

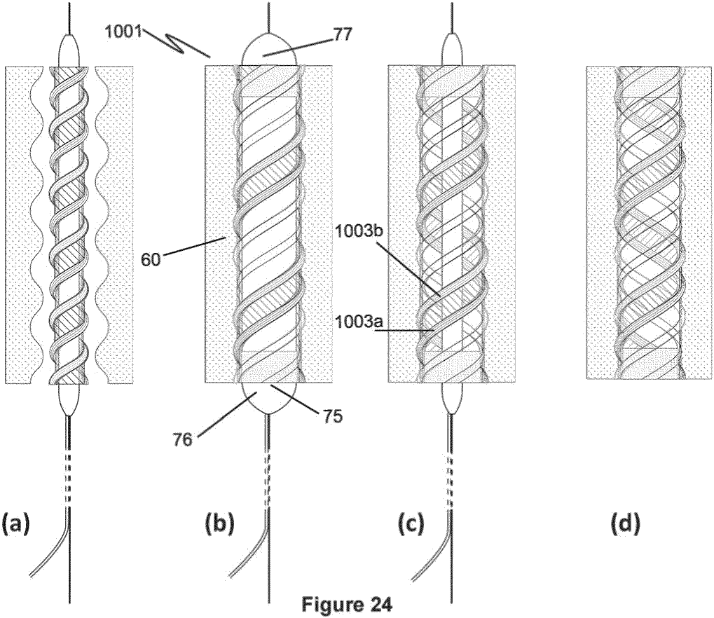

FIG. 24 shows a further embodiment of scaffold that may be deployed using an inflatable balloon (a) before inflation of the balloon, (b) after inflation of the balloon, (c) after deflation of the balloon, and (d) after the balloon has been removed.

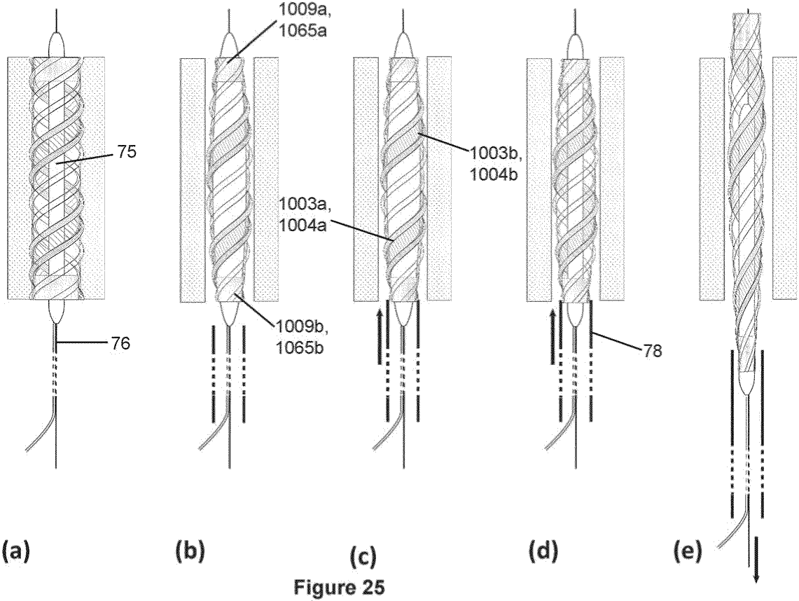

FIG. 25 shows the scaffold of FIG. 24 being retrieved wherein (a) a balloon is inserted into the scaffold, (b) the balloon is inflated, (c) heat from the balloon causes the internal handles to assume their pre-set shapes. (d) the balloon is partially deflated, and (e) the balloon and scaffold are removed.

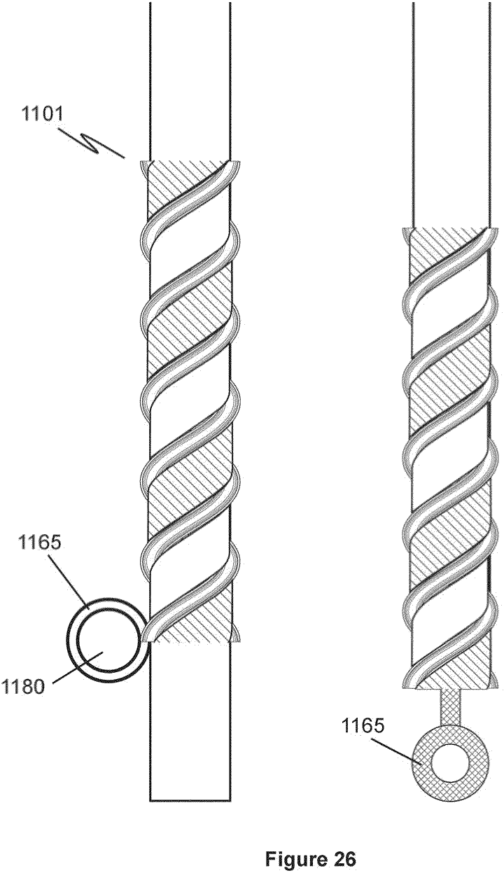

FIG. 26 shows a further embodiment of a scaffold as deployed in front elevation view, the scaffold having an anchorable handle.

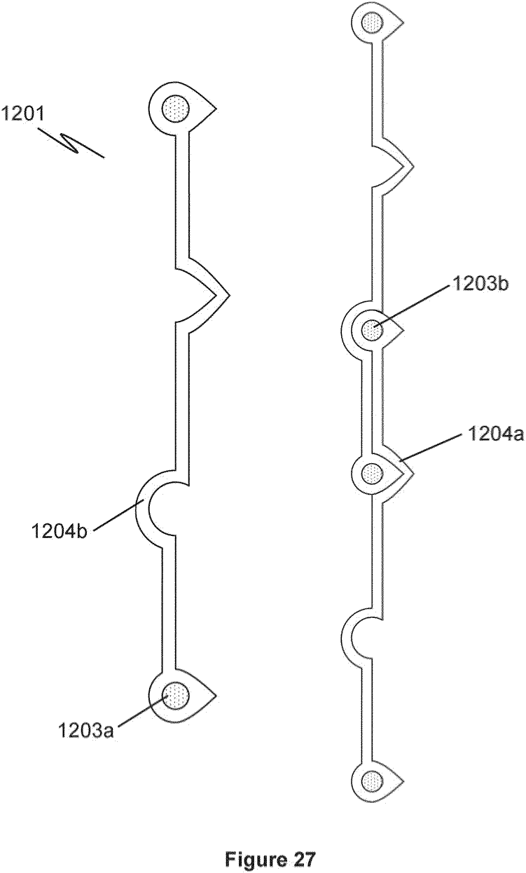

FIG. 27 shows a cross section of a further embodiment of a scaffold.

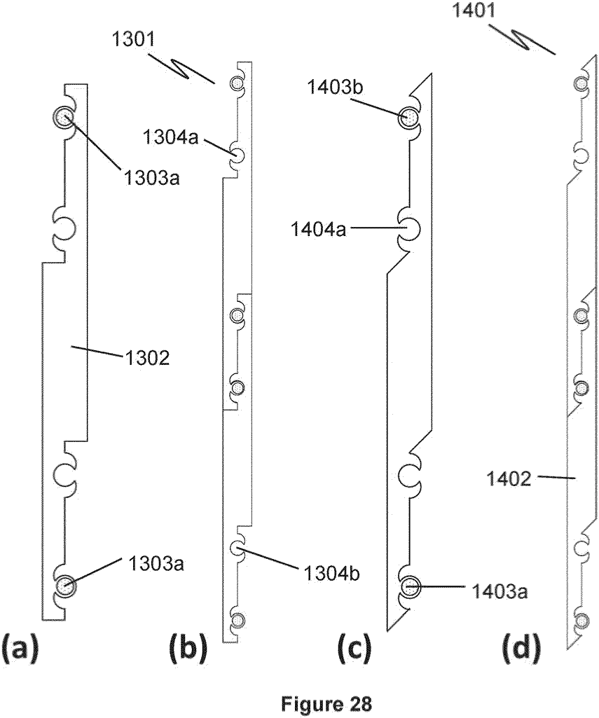

FIG. 28 shows (a) a cross section of a further embodiment of a scaffold. (b) a partial cross section of the scaffold when it is rolled and when the grooves and splines are engaged, (c) a cross section of a further embodiment of a scaffold, and (d) a partial cross section of the scaffold when it is rolled and when the grooves and splines are engaged.

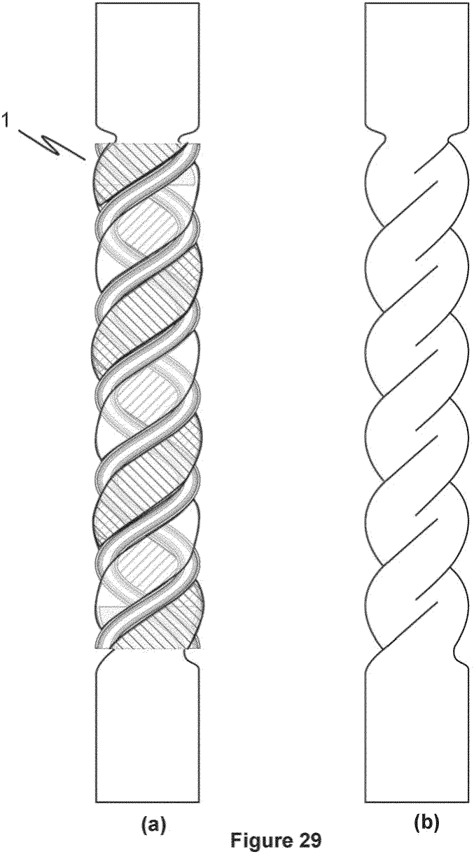

FIG. 29 shows (a) a front elevation view of the embodiment of a scaffold as shown in FIG. 1 in use, and (b) the configuration of an object when the scaffold is applied around the object.

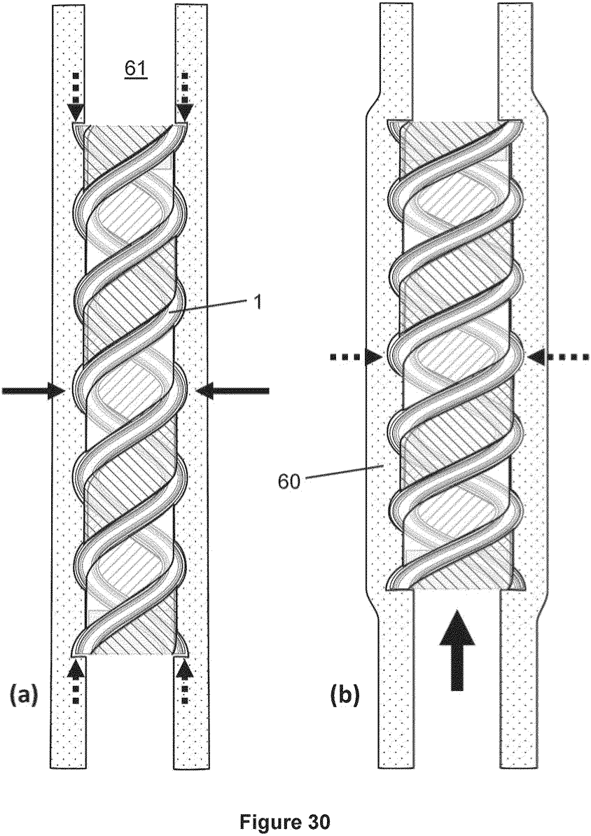

FIG. 30 shows (a) the scaffold of FIG. 1 in use deployed within a tubular object when radially compressed and (b) when longitudinally compressed.

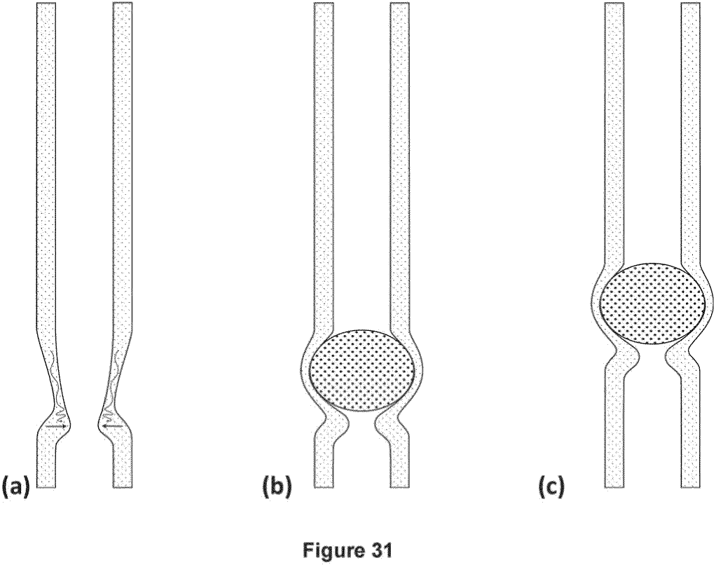

FIG. 31 shows a diagrammatic representation of peristalsis; (a) shows the direction of movement of muscles in the tubular biological object, (b) shows a body within the tubular object in a first position and, (c) shows the body moving through the tubular object via peristalsis.

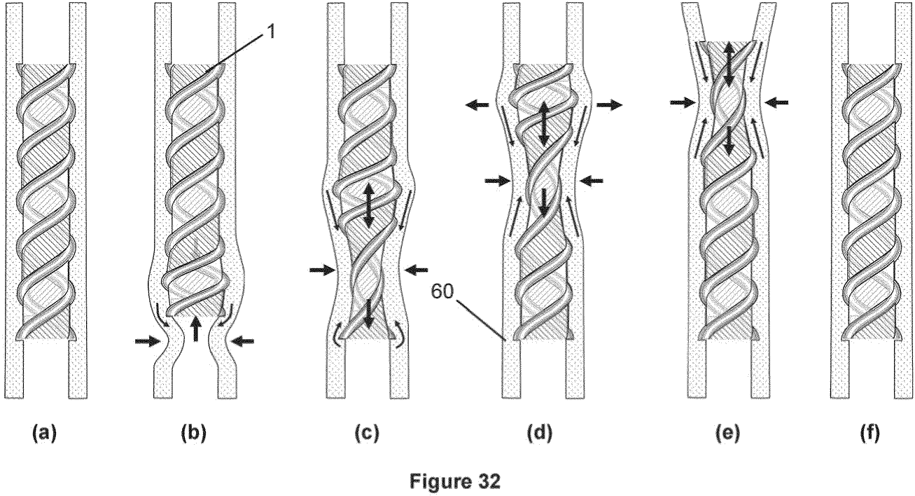

FIG. 32 shows a diagrammatic representation of peristalsis in a tubular object in which a scaffold is deployed: (a) before peristalsis. (b) beginning peristalsis, (c) - (e) the progression of peristalsis and, (f) after peristalsis.

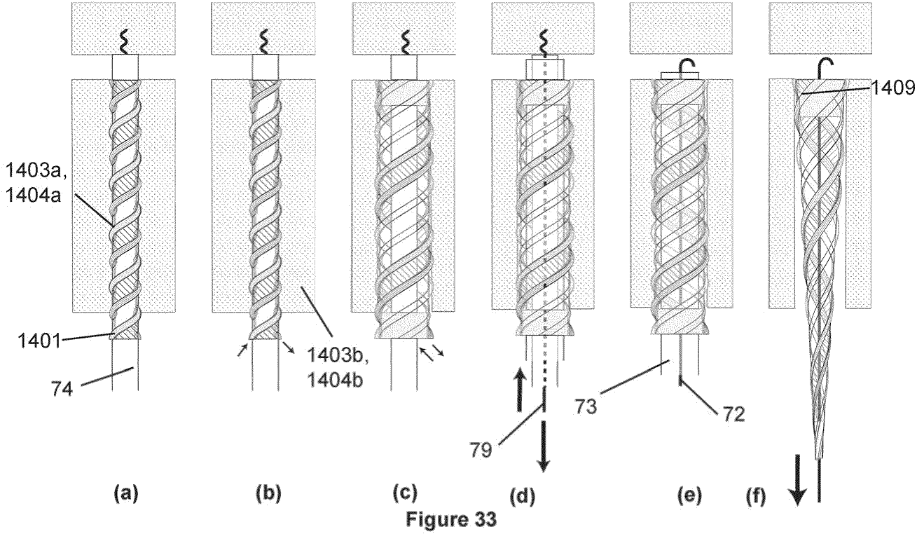

FIG. 33 shows a method of extracting a lead that is enveloped by a scaffold according to the invention; (a) shows the lead in situ, (b) when an electric current is applied, (c) after the electric current is applied, (d) sheath inserted around the lead. (e) guide wire inserted through sheath and lead removed and, (f) removal of the scaffold.

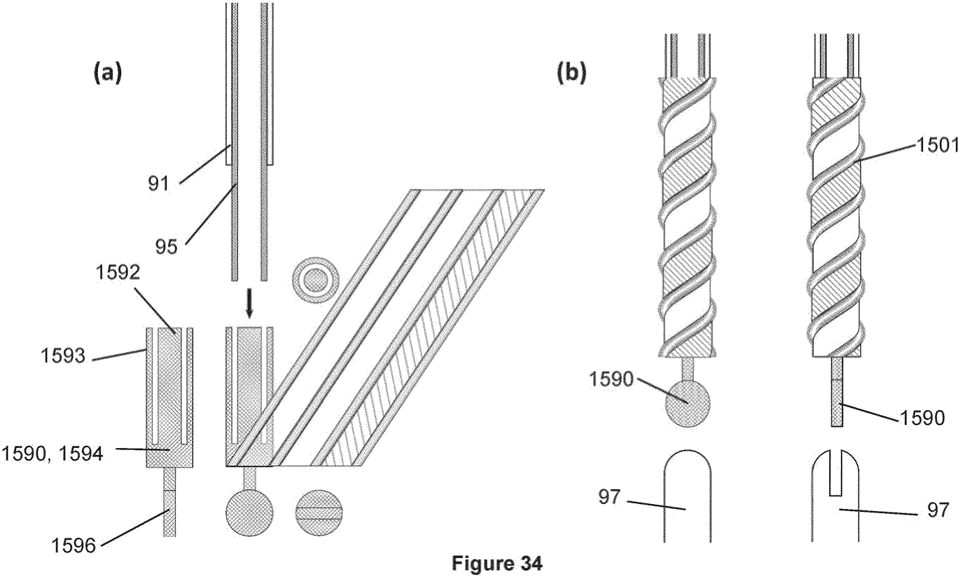

FIG. 34 shows a further embodiment of a scaffold used in conjunction with a plug for the pin of a transvenous lead in (a) exploded view and (b) applied to a lead.

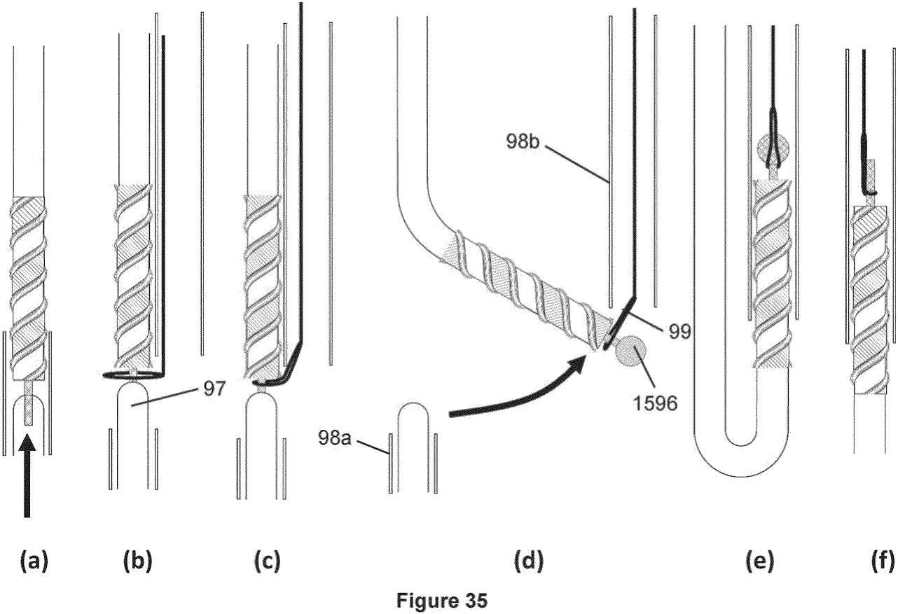

FIG. 35 shows the embodiment of FIG. 34 in use wherein (a) shows the scaffold and lead being passed through a first sheath by a ramrod dilator, (b) shows application of a snare to a handle of the plug, (c) and (d) show manipulation of the scaffold and lead via the snare to move it towards a second sheath and away from the ramrod dilator, (e) and (f) show drawing the lead through the second sheath via the snare.

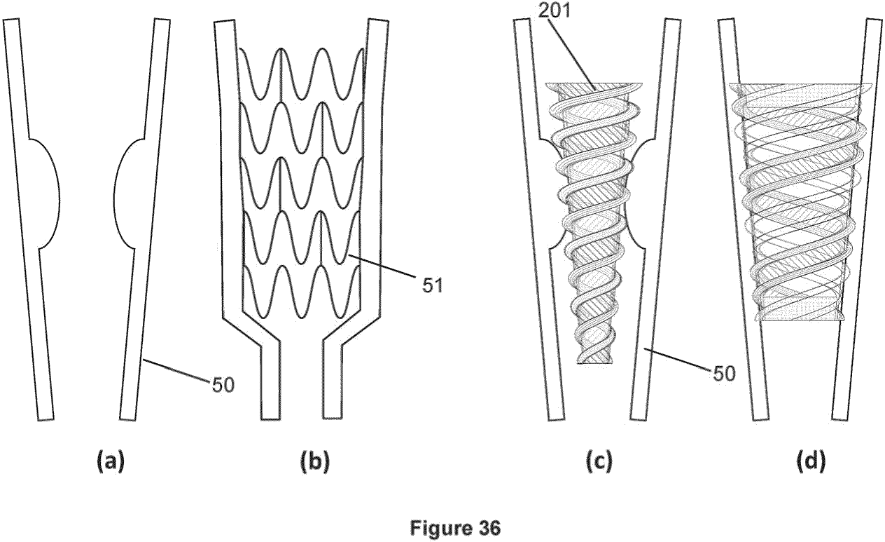

FIG. 36 shows (a) a tapered artery, (b) application of a known stent in a tapered artery and, (c) and (d) show application of a scaffold according to the invention in a tapered artery.

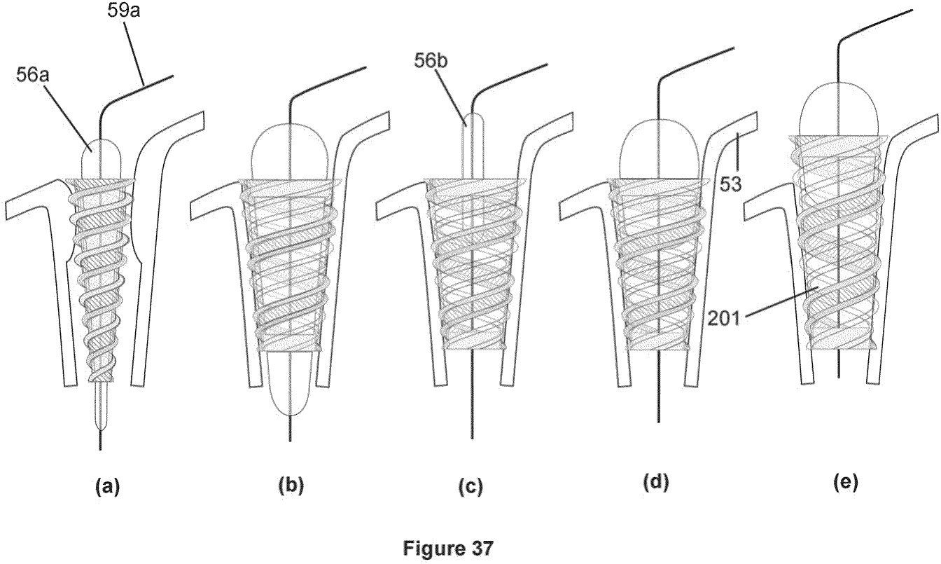

FIG. 37 shows a method of applying a conical scaffold at a slanted ostium where (a) shows initial deployment using a balloon and guide wire, (b) shows inflation of the balloon, (c) shows insertion of a second shorter balloon, (d) shows inflation of the second shorter balloon and, (e) shows the second shorter balloon being pulled out of the slanted ostium thereby drawing the scaffold out.

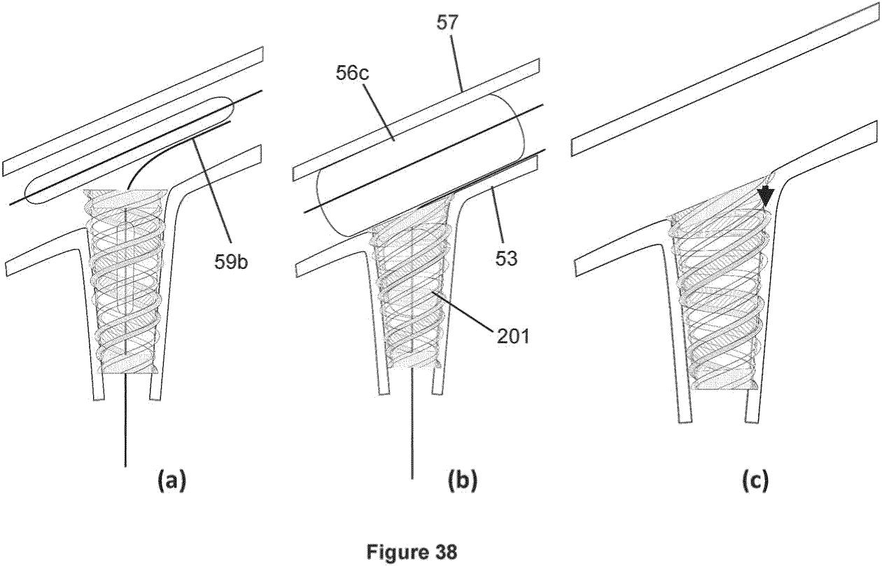

FIG. 38 shows a method of making the end of the scaffold flush with the slanted ostium where (a) shows deployment of a balloon along an opposing wall of the slanted ostium, (b) shows inflation of the balloon thereby moving the scaffold such that it is flush with the slanted ostium and, (c) shows the scaffold in the slanted ostium after removal of the balloons.

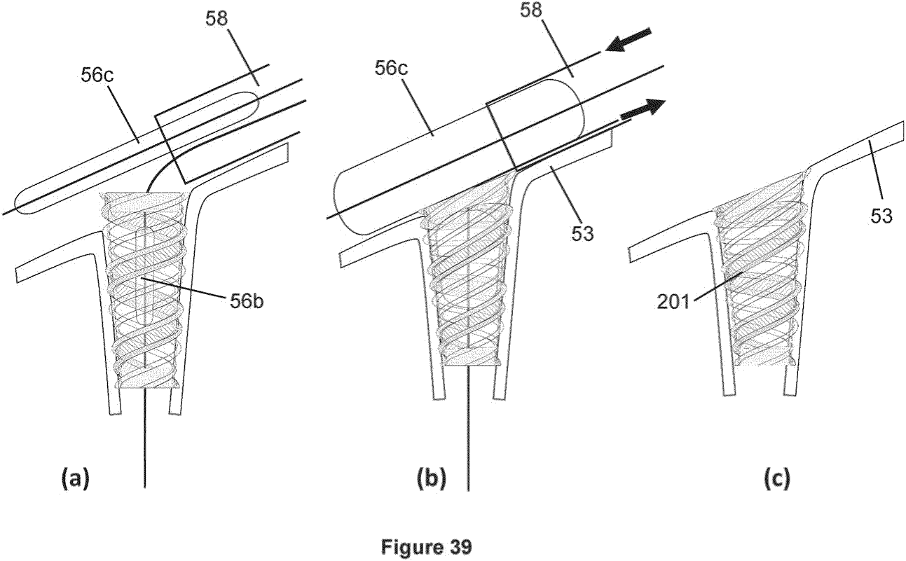

FIG. 39 shows a further method of making the end of the scaffold flush with the slanted ostium where (a) shows insertion of a guide catheter with a balloon to the end of the scaffold, (b) shows inflation of the balloon thereby moving the scaffold such that it is flush with the slanted ostium and, (c) shows the scaffold in the slanted ostium after removal of the balloons.

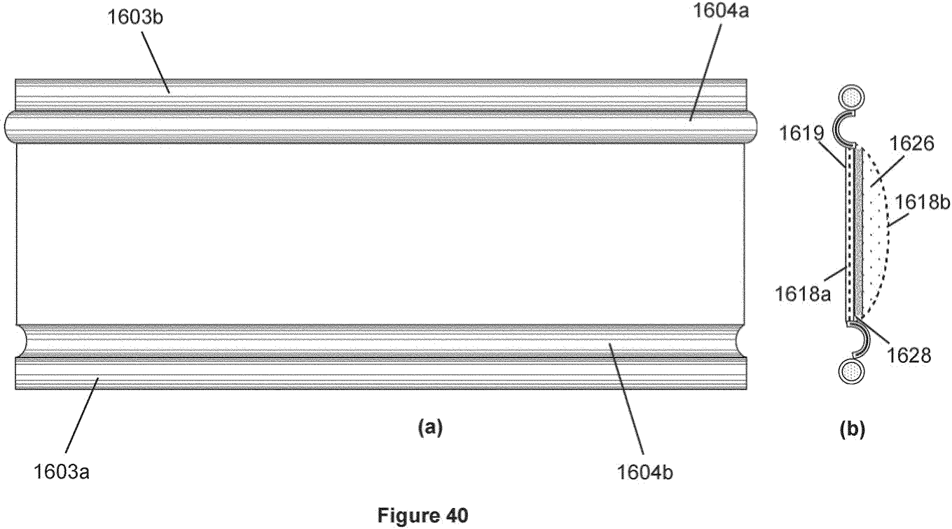

FIG. 40 shows (a) an elevation view of further embodiment of a scaffold in the flattened configuration and, (b) a cross sectional view of same.

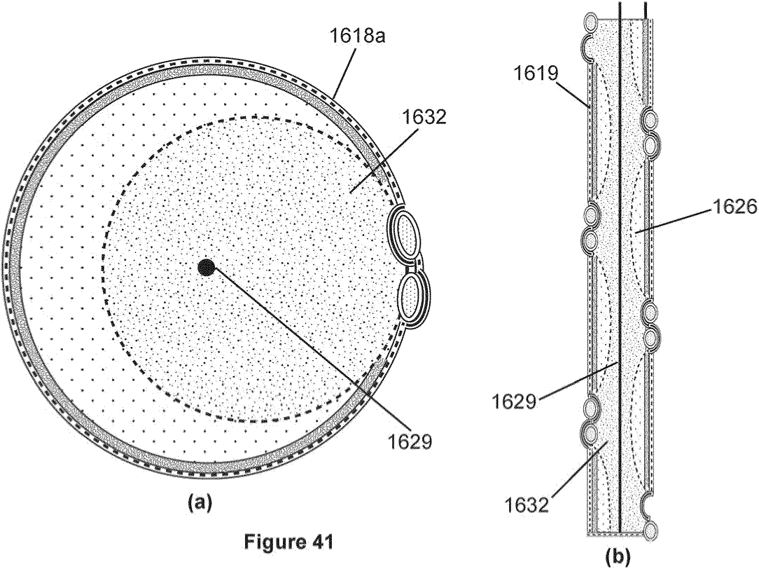

FIG. 41 shows (a) a cross sectional view of the scaffold of FIG. 40 when it is used to form a battery and (b) a transverse sectional view of same.

In FIG. 1 there is shown a first embodiment of a scaffold for a tube indicated generally by reference numeral 1. The scaffold has a membrane 2 and a pair of splines 3a, 3b that are embedded in the membrane 2 but could also be integrally formed with the membrane 2. The splines 3a, 3b are spaced apart from one another with the membrane 2 spanning therebetween. The membrane 2 further has a pair of grooves 4a. 4b adapted to receive the splines 3a, 3b when the membrane 2 is folded over on itself. The splines 3a, 3b are formed of shape memory materials, specifically nitinol, but could be formed of malleable materials. The grooves 4a, 4b may also be formed from shape memory or malleable materials. The splines 3a, 3b assume a pre-set helical shape spontaneously or in response to actuation.

The receiving grooves 4a, 4b match half of the profile of the splines 3a, 3b. whose cross section is circular, but may also be elliptical, rectangular, triangular or any other regular or irregular geometnc shapes in other embodiments. The scaffold 1 is made from a rectangular strip. In another embodiment as shown in FIG. 2,there is shown a scaffold 101 formed from a trapezoidal patch (bounded by curved rather than straight edges). The membrane 2 is a semi-rigid membrane, relatively resistant to stretching but amenable to bending. More complex geometric shapes of the scaffold membrane are possible depending on the practical uses and requirements. A trapezoidal scaffold 101 is generated by rotation of a spiral arm around an origin through an angle between two circular arcs of different radii or other spirals (FIG. 2). The most extreme spiral arm positions contain a pair of principal splines 103a, 103b; the in-between spiral arm positions contain one or more pairs of receiving grooves 104a, 104b indenting the two faces of the scaffold membrane 102 from opposite directions.

The principal splines 3a, 3b and/ or the receiving grooves 4a, 4b are made of either shape memory materials that will assume a pre-set helical (e.g. FIG. 1) or conical spiral (e.g. FIG. 2) shape (spontaneously or in response to actuation), or malleable materials that will retain the shape after non-elastic deformation. As shown in FIG. 12, the scaffold 1 may have an auxiliary spline or splines 9, 20. Instead of an auxiliary spline, a scaffold may have an auxiliary receiving groove that can receive an auxiliary spline of another scaffold. The scaffold may have an auxiliary spline and auxiliary receiving groove, two auxiliary splines, or two auxiliary receiving grooves.

In the rectangular scaffold 1, the pair of receiving grooves 4a. 4b indent the membrane 2 in opposing directions. In other words, when the scaffold 1 is flattened, one receiving groove 4a projects out of the plane of the membrane 2 in one direction, and the other receiving groove 4b projects out of the plane of the membrane 2 in the opposing direction. The width-wise distance from the first spline 3a to the first groove 4b is the same width-wise distance as that from the second spline 3b to the second groove 4a. The width of the membrane 2 can thereby be divided in the ratio k: (1 - k): k (0 < k < 1), FIG. 1).

Regarding the trapezoidal scaffold membrane 102, the angular distance from the first spline 103a to the first groove 104b is equal to that of the angular distance from the second spline 103b to the second groove 104a. Therefore, the angular width of the membrane 102 is divided in the ratio k: (1 - k): k, (0 < k < 1), FIG. 2). A scaffold may have several mirror pairs of receiving grooves. The base material of the scaffold membrane 2, 102 may be reinforced (and have other components bonded to it) by a laminating layer 10, 11 of stiffer materials (FIG. 1). The laminating layer 11 may have holes 11a punched into it at regular intervals in order to reduce the rigidity of the scaffold membrane and allow its easy perforation if necessary. A pair of auxiliary splines 9 (may also be made of materials with shape memory) and/ or handles (not shown) may also be incorporated into or attached to the scaffold to facilitate its deployment and retrieval. The scaffold membrane may be impregnated with perfluorocarbons (not shown) to achieve specific physical and chemical properties.

The principal and auxiliary splines and/or the receiving grooves (principal or auxiliary) need to be rigid enough to provide adequate mechanical support for and confer the required shape on the scaffold, but flexible enough to deform without breaking when an external force is applied. The splines and the grooves may be constructed out of a single material (e.g. a metal, an alloy, a polymer, a copolymer) or a composite of several materials (e.g. a metal alloy, a mixture of polymers, a polymer doped with inorganic compounds, a polymer reinforced with microfibrils of other materials, etc.). In the embodiment shown in FIGS. 1 and 2, the splines 3a, 3b, 103a, 103b, are formed from nitinol. Nitinol is a metal alloy which conducts electricity and possesses both superelasticity and heat-activated shape memory. Some polymers such as polylactic acid (PLA) have significant rigidity but limited elasticity and shape memory. After the external force used in deployment has been removed, the splines 3a, 3b and the grooves 4a, 4b will either retain the shape from non-elastic deformation (if their construction materials are malleable), or return to their pre-set shapes spontaneously or in response to actuation (if the construction materials have shape memory).

In the embodiment shown in FIG. 1, the scaffold membrane 2 is a laminate of layers of expanded polytetrafluoroethylene (ePTFE) 18a, 18b (flexible and flimsy) sandwiching a fluorinated ethylene propylene (FEP) core 19 (more rigid and tear resistant) with the receiving grooves directly moulded into it (FIG. 1). In another embodiment (not shown), the scaffold membrane is constructed of a single layer of a bioabsorbable polymer such as polylactic acid (PLA), poly-L-lactic acid (PLLA), poly-D-lactic acid (PDLA), poly-LD-lactic acid (PDLA), polyglycolic acid (PGA), poly(lactic-co-glycolic acid) co-polymer (PGLA), polycarprolactone (PCL), poly(glycolide-co-caprolactone) co-polymer (PGCL), polydioxanone (PDX) and polyorthoesters (POE), or combinations thereof. The thickness of the single layer is adjusted to give the mechanical properties functionally required for different sections of the scaffold membrane. The receiving grooves are directly moulded into the single layer. The principal and auxiliary splines, receiving grooves and handles may be fabricated as integral parts of the scaffold membrane if they are constructed of the same materials in one piece, or separately fabricated and then embedded into the scaffold membrane if they are constructed of different materials.

The principal and/or auxiliary spline-groove joints may be left bare or sealed with an adhesive (not shown) that may be rigid (e.g. a resin) or elastic (e.g. an elastomer) when set. The adhesive may be formed from a single component activated by pressure or by two components that are separately attached to or coated on to the principal splines and the receiving grooves, so that the adhesive only forms when the splines and the grooves come into physical contact and the curing process (if the adhesive is a two-part polymer) is activated. The adhesive may impart additional rigidity or flexibility and leak resistance to the scaffold.

The handles can be made of materials and into shapes and forms that will enhance the scaffold’s utility. The handles need to be attachable to the scaffold securely and relatively easily during manufacturing.

The “pores” of an ePTFE membrane can be made small enough to stop cell migration, and be impregnated with perfluorocarbons such as perfluoropolyether (PFPE), perfluoroperhydrophenanthrone (PFPH) or per-fluorodecalin (PFD) to produce a slippery liquid-infused porous surface (SLIPS) to prevent or reduce thrombosis, inflammation and bacterial adhesion. (Non-bioabsorbable fluoropolymer coated metal stents have been shown to be less thrombogenic and inflammatory than other drug-eluting stents covered with absorbable polymers.) A “drug-eluting” SLIPS can also be engineered to release molecules into the surrounding environment.

Referring now to FIG. 3, consider a length of a rectangular scaffold membrane strip cut obliquely across twice at the same angle to form a parallelogram, and oriented such that the principal splines forming the slanted sides and the cut edges forming the vertical sides, in the “standard” view (FIG. 3a). When transformed from the flattened configuration (FIG. 3a) to the rolled configuration (FIG. 3b), the lowermost spline 3b engages with the uppermost groove 4b. and the lowermost groove 4a engages with the uppermost spline 3a. If the scaffold strip 1 is rolled up parallel to its cut edges into a helix (FIG. 3b), the principal splines 3a, 3b will automatically fit into (and become locked in position by) the receiving grooves 4a, 4b further away (i.e. skipping the ones immediately adjacent), provided:

$$tan α = \frac{λ}{π d} \qquad \text{(1)}$$

$$w = \left(1 + k\right) λ cos α \qquad \text{(2)}$$

$${s}^{2} = {λ}^{2} + {π}^{2} {d}^{2} \qquad \text{(3)}$$

Where w is the width and k the “overlap” ratio of the strip: α the pitch angle, λ the pitch (longitudinal separation between successive turns of the same element of the scaffold), d the transverse diameter and s the length of one complete tum of the principal splines 3a, 3b and receiving grooves 4a, 4b in the resulting helical formation respectively (FIGS. 1 and 3). When equations (1) and (2) are fulfilled, successive turns of the scaffold membrane 2 (FIG. 4a) will telescope into each other (FIG. 4b), overlapping by a width of kλ along its longitudinal axis and forming a continuous surface of alternating single-layered 14 and double-layered 15 wall thickness (FIG. 4c). The telescopic cylindrical helix formation forms from a rectangular scaffold strip with vertical cut edges will have staggered and not flush ends.

Referring now to FIGS. 5 and 6 there is shown a further embodiment of a scaffold for a tube indicated generally by reference numeral 201. The scaffold may by substantially cylindrical or conical when in the rolled configuration. In the embodiment shown in FIGS. 5 and 6, the scaffold 201 forms a cone when in the rolled configuration. The scaffold 201 is substantially conical in the rolled configuration. In the rolled configuration, the grooves 204a-d and splines 203a, 203b are overlapping spiral helices. The cone has a narrow diameter end 212 towards the apex and a wide diameter end 213 at the base. The grooves 204a-d and splines 203a, 203b diverge in a direction from the narrow diameter end 212 towards the wide diameter end 213. The amount of membrane 202 between the splines and grooves increases in a direction from the narrow diameter end 212 towards the wide diameter end 213. In the rolled configuration, the cone is truncated and has a circular base. Specifically, the cone is a right circular cone. In the rolled configuration, the splines 203a, 203b and grooves 204a-d form overlapping spiral helices with a transverse diameter that decreases in a direction from the base of the cone to the apex. The scaffold 201 forms a right circular cone of base radius r and apex angle β (0 < β < n/2, FIG. 5) and can be “developed” by rolling up a circular sector of radius p, the slant height of the cone (FIG. 6):

$$ρ = r \text{cosec} β ⇔ r = ρ \text{sin} β \qquad \text{(4)}$$

If a circular arc subtends an angle θ in the cone’s base circle and an angle Φ in the developing circular sector, then:

$$ρ ⋅ ϕ = r ⋅ θ ⇒ ϕ = \frac{r}{ρ} θ = θ sin β ⇔ θ = ϕ \text{cosec} β \qquad \text{(5)}$$

Any point (r, θ, z) (in three-dimensional cylindrical co-ordinates) on a right circular cone with the apex at the origin, axis along the z axis and apex angle β:

$$z = r cot β = ρ cos β \qquad \text{(6)}$$

can be mapped (matched) continuously one-to-one to a point (ρ, Φ) (in two-dimensional polar co-ordinates) in the developing circular sector with the centre at the origin through equations (4) - (6), even if θ and Φ are allowed to take continuous values outside (0,2Π).

The scaffold 201 forms a telescopic conical helix formation when in the rolled configuration as shown in FIG. 5, and it is it is formable from a flat scaffold membrane patch cut out of a plane as shown in FIG. 6. The three-dimensional telescopic helix formation is constructed in (r, θ, 2) by rolling up a flat scaffold membrane patch cut out of a two-dimensional (ρ,ϕ) plane. A helix of uniform transverse diameter in the cylinder corresponds to a spiral of ever increasing (or decreasing) transverse radius in the cone.

If ƒ (ϕ) is a monotonic (strictly increasing or decreasing) differentiable function in ϕ, then

$$ρ \left(ϕ\right) = A f \left(ϕ\right) \qquad \text{(7)}$$

(A a scale factor) describes a spiral in the (ρ,ϕ) plane, which translates into another spiral:

$$r \left(θ\right) ⇒ ρ \left(ϕ\right) sin β = A sin β ⋅ f \left(θ sin β\right) \qquad \text{(8)}$$

in the (r,θ) plane and a conical spiral through equation (6). (The spiral in the (r,θ) plane is “shrunk” in size by a factor of sin β <1 and accelerated in rotational speed by a factor of cosec β > 1 compared to the spiral in the (ρ,ϕ) plane.)

The scaffold 201 relies on the splines 203a, 203b slotting into the receiving grooves 204a, 204b when the membrane 202 is rolled up into a telescopic conical helix formation. Suppose a principal spline 203a and its receiving groove 204a lying on the same radius (same angle Φ) in the developing circular sector, let their respective equations be:

$${ρ}_{0} \left(ϕ\right) = {A}_{0} f \left(ϕ\right) : {ρ}_{1} \left(ϕ\right) = {A}_{1} f \left(ϕ\right) \qquad \text{(9)}$$

A₀ < A₁ (i.e. the principal spline lies closer towards the origin than its receiving groove). In order that the principal spline 203a slots into its receiving groove 204a after a complete turn 2Π of the scaffold strip in θ (which corresponds to 2Π sin β in ø, equation (5)):

$$\begin{matrix}{ρ}_{0} \left(ϕ + 2 π sin β\right) = {ρ}_{1} \left(ϕ\right) ⇒ {A}_{0} f \left(ϕ + 2 π sin β\right) = {A}_{1} f \left(ϕ\right) \\ ⇒ f \left(ϕ + 2 π sin β\right) = \frac{{A}_{1}}{{A}_{0}} f \left(ϕ\right)\end{matrix} \qquad \text{(10)}$$

The ratio A₁ /A₀ is determined by 2π sin β and independent of ø and stays the same as ø varies. Replace 2Π sin β with φ and A₁/A₀ with g(φ) (φ and hence β are allowed to vary continuously):

$$f \left(ϕ + φ\right) = f \left(φ\right) ⋅ g \left(φ\right) \qquad \text{(11)}$$

Let ϕ = 0, then:

$$f \left(φ\right) = f \left(0\right) ⋅ g \left(φ\right) \qquad \text{(12)}$$

Substituting equation (12) into equation (11):

$$\begin{matrix}f \left(ϕ + φ\right) = \frac{1}{f \left(0\right)} f \left(ϕ\right) ⋅ f \left(φ\right) ⇒ \text{In} f \left(ϕ + φ\right) = \\ - \text{In} f \left(0\right) + \text{In} f \left(ϕ\right) + \text{In} f \left(φ\right)\end{matrix} \qquad \text{(13)}$$

For

$$p , q ∈ □$$

(natural numbers or positive integers), q> 0:

$$\begin{matrix}\text{ln} f \left(p ϕ\right) = - \text{ln} f \left(0\right) + p \text{ln} f \left(ϕ\right) \\ ln f \left(1\right) = ln f \left(q \frac{1}{q}\right) = - ln f \left(0\right) + q ln f \left(\frac{1}{q}\right) ⇒ ln f \left(\frac{1}{q}\right) = \\ \frac{1}{q} \left[ln f \left(1\right) + ln f \left(0\right)\right] \\ ln f \left(\frac{p}{q}\right) = - ln f \left(0\right) + p ln f \left(\frac{1}{q}\right) = \\ - ln f \left(0\right) + \frac{p}{q} \left[ln f \left(1\right) + ln f \left(0\right)\right]\end{matrix} \qquad \text{(14)}$$

∴ In ƒ(ϕ) is linear if ϕ is a rational number.

As ƒ (ϕ) is assumed to be differentiable. In ƒ (ϕ) is also differentiable where it is well defined. Thus

$$ln f \left(ϕ\right) = α + b ϕ \left(a , b \text{constants}\right) ⇒ f \left(ϕ\right) = {e}^{a + b ϕ} = {e}^{a} {e}^{b ϕ} = A {e}^{b ϕ} \qquad \text{(15)}$$

A conical scaffold 201 as shown in FIGS. 5 and 6 thus requires an underlying logarithmic spiral to work. The principal splines 203a, 203b and receiving grooves 204a, 204b in the developing circular sector as specified in equation (9) can then simply be slotted into one another by rotation around the origin.

A geometrical argument for the same deduction result works as follows. A spiral function ƒ(ϕ) can be transformed to cover the entire (p,ϕ) plane by either radial scaling ƒ(ϕ) ↦ A ƒ (ϕ) or angular rotation around the origin ƒ(ϕ) ↦ ƒ (ϕ + ψ). For the conical scaffold 201, these 2 families of spiral functions have to coincide, and one member can be transformed to another member by either scaling or rotation. For this to happen. the direction of the local tangent vector pρ + pϕϕ (p(ϕ)= A ƒ(ϕ)) needs to be scaling and rotation invariant (i.e. stays the same regardless of A and ϕ) and has a non-zero component in both directions (otherwise, one member cannot be transformed to another by both scaling and rotation, of circle) (FIG. 6):

$$\begin{matrix}\frac{\dot{ρ}}{ρ ϕ} = \frac{A d f}{A f d ϕ} = \frac{d f}{f} ⋅ \frac{1}{d ϕ} = b \text{constant} ⇒ \frac{d f}{f} = b d ϕ ⇒ ln f = a + b ϕ \\ ⇒ f = {e}^{a + b ϕ}\end{matrix} \qquad \text{(16)}$$

Equation (16) is the same as equation (15).

If a̅ = tan⁻¹ b is the “slanted” pitch angle of the telescopic conic helix formation (FIG. 6). then:

$$b = tan \tilde{α} \qquad \text{(17)}$$

If a̅ > 0, tan a̅ > 0, ƒ(ϕ) and ρ(ϕ) increase with ϕ (i.e. expanding spirals). If a̅ < 0, tan α̅ < 0, ƒ(ϕ) and p(ϕ) decrease with ϕ (i.e. contracting spirals).

Suppose the telescopic conical helix formation is to have a minimum transverse diameter d . Without loss of generality, let

[symbol]

$${ρ}_{0} \left(ϕ\right) = A$$

[symbol]

indicates text missing or illegible when filed
for the principal spline closest to the origin. and [symbol] correspond to the point on the telescopic conical helix formation with the smallest transverse radius.

$${r}_{0} \left(0\right) {A}_{0} sin β = \frac{d}{2} ⇒ {A}_{0} = \frac{d}{2 sin β} \qquad \text{(18)}$$

$$∴ {ρ}_{0} \left(ϕ\right) = \frac{d}{2 sin β} {e}^{ϕ sin d} \qquad \text{(19)}$$

$${r}_{0} \left(θ\right) = {ρ}_{0} \left(ϕ\right) sin β = \frac{d}{2} {e}^{ρ sin β t \text{an} d} \qquad \text{(20)}$$

[symbol]

indicates text missing or illegible when filed
If a receiving groove and the remaining principal spline is phase shifted from the lowest principal spline by ψ in θ, the slant height ρ₁ (ϕ) is given by: [symbol] For one complete turn in the telescopic conical helix formation (i.e. ψ = 2Π): the overlap ratio k : [symbol] While the overlap ratio κ stays constant if the apex angle β and the slanted pitch angle α̅ stay constant, the width of membrane overlap ρ(ϕ+2Πsinβ)-ρ(ϕ) = ρ(ϕ)(κ-1) varies with ϕ. Regardless of whether α̅ > 0 or α̅ < 0 (and ρ is an expanding or contracting spiral), membrane overlap is wider at the wide end of the telescopic conical helix formation.

A transverse cut across the cone at slant height p₀ corresponds to a circular arc of radius p₀ in the (p,ϕ) plane. If [symbol] and [symbol] the trapezoidal patch bordered by the 2 circular arcs p = ρ₀ and ρ = ρ₂ (ϕ:ϕ₀ → ϕ₀ + ø₁) and the 2 logarithmic spirals [symbol] and [symbol] will give rise to a telescopic conical helix formation truncated transversely at both the apical and base ends (FIG. 6).

Referring now to FIG. 7 there is shown the rolled configuration of a scaffold 1 of FIG. 1. In this configuration, the splines 3a. 3b and grooves 4a, 4b form helices whose turns directly stack on one another in the neutral unbent state. Like helical springs, the splines 3a, 3b and the spanning membranes 2 can accommodate bending of the helix formation by twisting along their own longitudinal axes, which does not involve or require changes in their dimensions. The decrease/increase in length on the inner/outer curvature of the bend can be achieved by compression/extension of the receiving grooves 4a, 4b (FIG. 7). Even though the splines 3a, 3b may no longer be locked in the receiving grooves 4a, 4b, the in-built functional redundancy in the scaffold 1 (i.e. overlap of the spanning membranes) ensures a continuous surface is still maintained for a telescopic helix formation. (A scaffold strip or patch with no in-built membrane overlap, and only one principal spline and one receiving groove along the two long edges, will develop gaps in the surface of the helical formation on the outer curvature of the bend.)

Referring now to FIG. 8. there is shown a further embodiment of a scaffold according to the invention referred to generally by reference numeral 301. The scaffold 301 is cylindrical in the rolled configuration and has spaced apart splines 303a, 303b, with two pairs of receiving grooves 304a-d therebetween. In this embodiment at least one pair of receiving grooves 304c, 304d will not be occupied by the principal splines 303a, 303b in the telescopic helix formation (FIG. 8). The unoccupied receiving grooves 304c, 304d can absorb the compression on the inner curvature and extension on the outer curvature of the bend so that the principal splines 303a, 303b can stay in place in the occupied receiving grooves 304a. 304b.

In use when supporting a tubular object, the scaffold 1, 101, 201, 301. spreads any bend to which the tubular object may be subjected over a longer longitudinal span. The reduction in curvature protects the tubular object and its contents from fatigue fracture.

Referring now to FIG. 9, there is shown two scaffolds 401, 501. The scaffolds 401, 501 have the same area, but due to differing pitch angle, when rolled they produce tubular scaffolds of differing diameters and differing longitudinal length. For any given rectangular scaffold strip construction (i.e. w and k fixed), multiple telescopic cylindrical helix formations are possible depending on pitch angle α . For a paralleiogram scaffold membrane strip with the principal splines measuring S in length (fixed area Sw regardless of pitch angle α, FIG. 9), the total longitudinal span L of a principal spline is given by:

$$L = S sin α = \frac{S w}{\left(1 + k\right) π d} = \left(1 + k\right) L d = \frac{S w}{π} \qquad \text{(23)}$$

By equation (23), for the same area of the regular scaffold strip Sw, the longitudinal span L, transverse diameter d and overlap ratio k (through the term l+k) are inversely related. If the overlap ratio k is also fixed, longitudinal deformation of the scaffold will result in and can only occur in the presence of opposite concomitant radial deformation (i.e. Poisson effect).

For the trapezoidal scaffold 201 (determined by A₀ and b in equations (17) and (18); as shown in FIG. 6), multiple telescopic conical helix formations (corresponding to different apex angles β) are also possible. Only the ratio d/sin β, and not the absolute values of d and sin β, is fixed by the value of A₆ . The scaffold strip is able to reduce its transverse radius, increase the number of turns within and increase its longitudinal span (given by Pₘₐₓ cos β, ρₘₐₓ the maximum slant height and a fixed property of the membrane patch) by winding up more tightly into a narrower cone. Radial contraction and longitudinal expansion occur simultaneously and are inseparable (i.e. the Poisson effect). The slanted pitched angle α̂, which is fixed by the value of b = tan α̅, stays the same.

The practical implications of the inverse relationship between longitudinal and radial deformations of the scaffold are:
i. Extending the longitudinal span of the scaffold will reduce it into a smaller transverse diameter (which may be useful for minimal access deployment and retrieval).
ii. Extending the longitudinal span of the scaffold deployed on the outside of a tubular object will decrease its transverse diameter and enhance its radial grip on the tubular object.
iii. Fixing the longitudinal span of the entire scaffold (i.e. preventing it from extending) deployed on the inside of a tubular object will protect any segment of it against radial compression (i.e. radial compression on a segment of the scaffold is distributed along its entire longitudinal span.)
iv. Contracting the longitudinal span of the scaffold deployed on the inside of a tubular object will increase its radial expansion against the wall.

Extending the scaffold does not only reduce its transverse diameter but also distributes its physical bulk over a longer longitudinal span, which makes the scaffold more flexible and deliverable along a tortuous anatomical course.

Radial expansion without longitudinal shortening (i.e. Poisson ratio = 0) of a telescopic cylindrical helix formation can be achieved by winding the scaffold membrane into a tighter roll and then unwinding it dunng deployment. Referring now to FIG. 10, the scaffold 1 can be modified by either attaching triangular flaps 22 to the vertical cut edges of the parallelogram scaffold membrane strip. or such flaps 22 can be integrally formed in the scaffold 1 during manufacture, for example, by forming horizontal cut edges as opposed to vertical cut edges. In either case, the flattened configuration thereby has horizontal cut edges 21 (FIG. 10). Auxiliary splines 20 in the form of rolled-up strips with channels 24a, 24b to accommodate the principal splines 3a, 3b at the two ends and indentations 25a, 25b to match the receiving grooves 4a, 4b can be attached to one (FIG. 11) or both (FIG. 12) of the horizontal cut edges. A telescopic helix formation rolled up from such a scaffold strip will have flush rather than staggered ends (FIG. 13).

The auxiliary splines 9 can also take the form of a wire 30 (circular, elliptical, rectangular or other geometric shape in cross-section) at one end and a matching receiving groove 31 at the other (FIG. 14). The wire auxiliary spline 30 is pre-shaped into a cylindrical helix with the same transverse diameter as that formed by the principal splines 3a, 3b and can slot into the auxiliary receiving groove 31 of an adjacent identical scaffold 1 if several of them are deployed in series (FIG. 15).

Referring now to FIG. 16 there is shown a further embodiment of a scaffold 601. The scaffold 601 has a first rolled configuration (FIG. 16(a)) and a second rolled configuration (FIG. 16 ((b)) wherein in the second rolled configuration, the scaffold 601 is twice the diameter than the first rolled configuration. From equation (23), for a given area of scaffold strip Sw, the longitudinal span of the scaffold will stay the same if the term (1 + k)d remains constant. If a scaffold membrane is equipped with two pairs of mirror receiving grooves corresponding to overlap ratios k and k′, then 2 telescopic cylindrical helix formations with the same longitudinal span but different transverse diameters d and d′ are possible:

$$\frac{{d}^{′}}{d} = \frac{1 + k}{1 + {k}^{′}} \qquad \text{(24)}$$

As k → 1 and k¹ → 0, the maximum radial expansion that can achieved by this method is x2. For example, scaffold 601 has splines 603a, 603b and two pairs of grooves 604a-d. In the first rolled configuration, the splines 603a, 603b are engaged with the central grooves 604c, 604d that are furthest away from the splines 603a, 603b when in the flattened configuration Specifically, spline 603a is engaged with groove 604c, and spline 603b is engaged with groove 604d. The other grooves 604a, 604b remain free. In the second rolled configuration, the splines 603a, 603b are engaged with the grooves 604a, 604b that are adjacent to the splines 603a, 603b in the flattened configuration. Specifically, spline 603a is engaged with groove 604a, and spline 603b is engaged with groove 604b. Grooves 604c and 604d remain free.

Referring now to FIG. 17 there is shown an embodiment of a scaffold indicated by reference numeral 701. The scaffold 701 is formed from repeating units, wherein across the width of each unit there is a part of a receiving groove 704, a span of membrane 702, an entire receiving groove 704. a further span of membrane 702. and then a further part of a receiving groove 704. To achieve a higher radial expansion ratio, the scaffold 702 is widened (in the flattened configuration) to have n (a positive integer) repeats of units. If p (p a positive integer; p ≤ n) units are used to form one turn of the telescopic cylindrical helix formation at constant pitch angle α, the transverse diameter d will be:

$$d = p \frac{u}{π sin α} ∝ p \qquad \text{(25)}$$

where n is the width of a unit. The pitch angle α stays the same for the different transverse diameter telescopic helix formations.

The diameter of the telescopic cylindrical helix formation is smallest when p = 1 and largest when p = n, The radial expansion ratio possible of the scaffold takes the form p/q, where p>q are positive integers ≤n . The widened scaffold strips and telescopic helix formations possible for n = 2 and n = 3 are shown in FIGS. 18 and 19 respectively

Referring now to FIG. 20 there is shown a scaffold 801 foldable into a conical formation and having either a wire auxiliary spline/groove arrangement 830, 831 or flat strip auxiliary splines 820. A trapezoidal scaffold such as that shown in FIG. 20 can also have multiple pairs of mirror receiving grooves subtending the same angular width. The radial expansion ratio possible of the scaffold takes the form p/q. where p>q are positive integers ≤n. Radial expansion/ compression is however accompanied by concomitant longitudinal compression/ expansion (i.e inevitable Poisson effect).

Radial expansion without longitudinal shortening through axial unwinding allows the scaffold for telescopic cylindrical helix formation to be positioned precisely in a collapsed state at the target site before deployment. However, the wound-up scaffold has increased physical bulk compacted into a smaller volume and may become stiffer and less deliverable along a tortuous course.

The scaffold 1 can be deployed simply by unwinding it and then wrapping it around a tubular object turn by turn, so that the splines 3a, 3b fall within their receiving grooves 4a, 4b. If the scaffold 1 is pre-shaped to a helical formation with a transverse diameter slightly smaller than the outer diameter of the tubular object, elastic recoil will ensure a good radial grip by the scaffold 1 and fix its position on the tubular object. If necessary, the scaffold 1 can be extended longitudinally along the tubular object, so that its transverse diameter will decrease and the scaffold 1 will grip the tubular object more tightly (FIG. 21). The scaffold 1 can be removed from the extemal surface of the object by unwinding it turn by turn, starting from the outermost one.

Assuming the scaffold 1 is applied to the outside of the tubular object 60 in the distal (further away from the user) to proximal (closer to the user) direction, the distal end of the next turn will be external to the proximal end of the last turn, the “proximal-external-to-distal” topology (FIG. 22a).

If the scaffold 1 is to line the inner surface (lumen) of the tubular object 60, it needs to have a transverse diameter larger than the diameter of the object’s lumen so that it can be held in place by friction against and/ or distortion of the lumen’s wall. Before the scaffold 1 can be introduced into the lumen of the object 60 through either of its ends or an opening on its side, the scaffold 1 needs to be collapsed into a transverse diameter smaller than the lumen’s. When the scaffold is retrieved out of the tubular object 60, it needs to be reduced back into a smaller transverse diameter.

Ideally, the deployment and retrieval apparatus (external tools and attachments to the scaffold 1) should be physically as small as possible. However, if the apparatus is too small, they may be challenging to handle and not strong enough to manipulate the scaffold 1 with. If the apparatus is too large, it may cause obstruction for the scaffold or be too bulky to be delivered to the target site through minimal remote access. The scaffold 1 is designed with dedicated features to enable lower profile delivery and easy reliable atraumatic, nondestructive retrieval through minimal access.

Assuming the scaffold 1 is applied to the tubular object 60 in the distal (further away from the user) to proximal (closer to the user) direction, the distal end of the next turn needs to be internal to the proximal end of the last turn, the “proximal-internal-to-distat” topology (FIG. 22b).

Referring now to FIG. 23, there is shown an embodiment of a scaffold 901 in which the scaffold 901 has handles 965a, 965b. wherein one handle 965a is disposed at one longitudinal end of the scaffold 901 when in the rolled configuration, and the other handle 965b is disposed at the opposing longitudinal end. The scaffold 901 can be directly manipulated with grasping tools 68a, 68b (e.g. snares, forceps, catheters) The scaffold 901 can be longitudinally extended to achieve radius reduction (FIG. 23a). The distal end of the extended scaffold 901 is positioned to the target site by the grasping tool and then held stationary. The proximal end of the extended scaffold 901 is then gradually brought into position (FIG. 23b). The scaffold 901 may be deployed in an elastic state and will assume the pre-set shape spontaneously. Alternatively, the scaffold 901 may be deployed in a malleable state and is activated to assume its pre-set shape by a stimulus. If the principal splines 903a, 903b are made of nitinol, they can be activated to assume their pre-set shapes by heat applied directly through the grasping tools or generated by ohmic heating of the splines 903a, 903b by passing electric currents across the principal splines 903a. 903b between the grasping tools 68a, 68b. The grasping tools 68a, 68b can be used to adjust the scaffold’s deployment site more precisely as it gradually assumes its pre-set shape. Once the scaffold 901 has been positioned at the target site, the grasping tools 68a, 68b are released and removed, leaving the scaffold 901 in place (FIG. 23c). During retrieval, the handle 965b on the proximal end of the scaffold 901 is regrasped (FIG. 23d) and pulled, extending the scaffold’s longitudinal span and reducing its radius (FIG. 23e). The proximal-internal-to-distal topology allows the deployed scaffold 901 to be disassembled safely (no force on the tubular structure’s wall) and easily turn by turn,

The most difficult step in scaffold retrieval is likely to be re-grasping the handle. If necessary, the grasping tool 68a can be left permanently in place (FIG. 23d) as a “mooring line” to prevent scaffold migration post deployment, and as a “fishing line” to pull the deployed scaffold 901 in during retrieval.

Referring now to FIG. 24 there is shown a further embodiment of a scaffold 1001 wherein the splines 1003a, 1003b are formed from malleable material, and wherein the scaffold 1001 and is deployable by a balloon 75. In use, the scaffold 1001 is wrapped around an inflatable balloon 75 in a collapsed state with a reduced transverse diameter but the intended longitudinal span (as in standard angioplasty techniques used in medical vascular interventions). The wrapping of the scaffold 1001 around the inflatable balloon 75 is from the shaft end 76 towards the tip end 77, in order so that the tip end-external-to-shaft end topology of the scaffold 1001 with respect to the balloon 75 becomes the proximal-internal-to-distal topology with respect to the tubular object 60 into whose lumen the scaffold 1001 will be placed. The spacings between the splines 1003a, 1003b and receiving grooves can be chosen such that the collapsed scaffold will grip securely on to the external surface of the uninflated balloon (75; FIG. 21) and will not become detached when it is positioned to the target site. (Stent embolisation before deployment can occur during medical vascular intervention.)

Once positioned at the target site (FIG. 24a), the balloon 75 is inflated (FIG. 24b). The scaffold 1001 radially expands while maintaining the longitudinal span by unwinding (see also FIG. 18). The balloon 75 is deflated (FIG. 24c) and withdrawn out of the deployed scaffold 1001, leaving it at the target site (FIG. 24d).

If the scaffold 1001 is made of malleable materials with no shape memory components, the scaffold 1001 relies on permanent non-elastic deformation of its components and the spline-groove locks to maintain shape after deployment. Alternatively, the scaffold may contain shape-memory materials and be mounted on the balloon 75 in a malleable state. Shape memory activation (e.g. by heat) is achieved by either using warm liquid (e.g. radio-opaque contrast warmed up to the transition temperature of the shape memory materials) or passing an electric current between the ends of the collapsed scaffold through electrodes on the balloon catheter or the guide wire passing through it.

The scaffold 1001 has two internal handles 1065a, 1065b at either longitudinal end of the scaffold 1001. The handles 1065a, 1065b are formed from auxiliary splines 1009a, 1009b made of shape memory materials such as nitinol. For retrieval, an inflatable balloon 75 with a transverse diameter the same as but a longitudinal span shorter than the deployed scaffold 1001 is inserted through its central lumen (FIG. 25a). The balloon 75 is inflated to contact the deployed scaffold 1001 with a liquid (radio-opaque contrast) warmed up to the transition temperature of the auxiliary splines 1009 (FIG. 25b). Heat is transmitted by conduction to the principal splines 1003a, 1003b and possibly also the receiving grooves 1004a, 1004b. The auxiliary splines 1009a, 1009b form the internal handles 1065a, 1065b of the scaffold 1001 and return to their pre-set shapes, which are circular rings with a diameter smaller than that of the inflatable balloon 75 (FIG. 25b). The guide catheter 78 used to pass the balloon 75 is advanced to “capture” the “gathered in” (like with a purse string) proximal end of the deployed scaffold 1001 (FIG. 25c). (The guide catheter 78 has a diameter larger than that pre-set for the handles 1065a, 1065b) The balloon 75 is then partially deflated such that its diameter is smaller than the transverse diameter of the telescopic cylindrical helix formation determined by the principal splines 1003a, 1003b but larger than the transverse diameter of the circular ring determined by the auxiliary splines 1009a, 1009b (FIG. 25d). The partially inflated balloon 75 acts as a “plug” to pull the proximal auxiliary spline 1009b and then other parts of the scaffold 1001 into the guide catheter 78 (FIG. 25d). The proximal-internal-to-distal topology ensures the deployed scaffold 1001 can be disassembled turn by turn easily when pulled proximally.

Only the proximal auxiliary spline 1009b is needed for scaffold retrieval by the method depicted in FIG. 25. The distal end of the scaffold 1001 does not necessarily need to be equipped with an auxiliary spline 1009a, 1009b for this method to work. However, having auxiliary splines 1009a, 1009b as internal handles 1065a, 1065b at both ends of the scaffold 1001 allows the stent to be retrieved in both directions. This may be practically advantageous in certain situations (For example, an aortic stent-graft may be retrieved from either the femoral or the subclavian/ brachial/ radial approach.) External and internal handles can be complementary and do not need to be mutually exclusive. A scaffold may use external handles for deployment (as in FIGS. 23a and 23b), and internal handles for retrieval (as in FIG. 25). The external handles may be detached from the scaffold as soon as it has been deployed to avoid any permanent obstruction to luminal flow.

Referring now to FIG. 26, there is shown a further embodiment of a scaffold indicated by reference numeral 1101. The scaffold 1101 has a handle 1165 that is can be anchored to an adjacent structure (e.g. by sutures or other fixation mechanisms, FIG. 26). The handle 1165 has an aperture 1180 to receive a suture.

Referring now to FIG. 27 there is shown a further embodiment of a scaffold indicated by reference numeral 1201. The splines 1203a, 1203b have a teardrop shaped cross-section and the grooves 1204a, 1204b are correspondingly shaped to receive the splines 1203a, 1203b, with the cross-section of one groove 1204a corresponding to the pointed end of the teardrop shaped spline 1203a, and the other groove 1204b being shaped to correspond to the rounded end of the teardrop shaped spline 1203b. In FIG. 28 there is shown an embodiment of a scaffold indicated by reference numeral 1301. The scaffold 1301 has a spline-groove arrangement wherein the splines 1303a, 1303b project from the surface of the membrane 1302 and have spaces to either lateral side of the spline 1303a. 1303b to receive the groove 1304a, 1304b, which envelopes the spline 1303a, 1303b at either lateral side thereof. Furthermore, in the flattened configuration, the thickness of the membrane 1302 is greater between the grooves 1304a, 1304b than it is between the spline 1303a, 1303b and grooves 1304a, 1304b. More specifically, the membrane 1302 is twice as thick in the part of the membrane 1302 between the groves 1304a, 1304b than between the splines 1303a, 1303b. When rolled, the membrane 1302 then has a consistent thickness. The membrane 1302 has a consistent thickness from the spline 1303a to the first groove 1304a and thereafter it extends orthogonally from the surface of the membrane 1302 to double in thickness. In the embodiment shown in FIGS. 28(c) and 28(d), the scaffold 1401 is similar to the scaffold 1301, except the membrane 1402 gradually slopes up from the first groove 1304a to double in thickness. This strengthens the scaffolds and reduces/ prevent leakage through the spline-groove joints.

If the object to which the scaffold 11 is applied externally is sufficiently flexible, the telescopic (cylindrical or conical) helix formation of the scaffold 11 may be able to distort the object into a helical formation as well (FIG. 29). The net result is two intertwined helical formations which will be very resistant to relative longitudinal displacement between the two. Shape distortion of the object is a novel mechanism of preventing it from sliding in and out of the grip of a scaffold 1.

Referring again now to FIG. 22, if the tubular object within which the scaffold 101 is deployed carries a fluid flow in a direction (proximal-to-distal) opposite to that in which a scaffold 1 is deployed (distal-to-proximal), the proximal-internal-to-distal topology will produce a “roof-tile” effect and reduce or even prevent leakage of the fluid through gaps between the turns of the scaffold 1.

Referring now to FIG. 30, radial contraction of the scaffold 1 can only occur if concomitant longitudinal extension is permitted. Radial compression on a segment of the scaffold 1 will not result in a reduction in its transverse diameter if the scaffold 1 is prevented from longitudinally extending provided its purchase on the wall of the tubular object 60 does not slip (FIG. 30a). In one sense, the longitudinal integrity of the tubular object 60′s wall is recruited by the scaffold 1 to combat radial compression by it. Compared to stents with independent circumferential rings longitudinally linked together, radial compression on a segment of the scaffold 1 is distributed along its entire longitudinal span.

Suppose the scaffold 1 is deployed in a tubular object 60 with the proximal-intemal-to-distal topology and the tubular object 60 has a proximal-to-distal fluid flow in its lumen 61, the flow will tend to wash a scaffold 1 turn distally into the next turn, wedging it open wider (FIG. 30b). The rise in the transverse diameter gives rise to a stronger radial compression on the scaffold 1. The action (longitudinal push) and reaction (radial compression) forces are positively correlated and may completely cancel out each other, reducing the chance of scaffold 1 dislodgement. With this adaptive counter-dislodgement mechanism, the scaffold 1 does not need to hugely oversized or have a very high permanent resting radial expansion pressure, reducing the risk of damage to the wall of the tubular object 60.

Referring now to FIG. 31 there is shown a diagrammatic representation of peristalsis. Peristalsis is a wave of segmental radial constriction and longitudinal shortening that sweeps in the proximal to distal direction of a tubular biological object. Radial constriction is mediated by contraction of the circular smooth muscles lining the tubular object and prevents the luminal contents from moving in distal-to-proximal direction (FIG. 31a). Longitudinal shortening happens just distal to radial constriction and is mediated by contraction of the longitudinal smooth muscles also lining the wall of the tubular object. The longitudinal shortening “pulls” the more distal segment of the tubular object’s wall proximally over the luminal contents, effectively moving the contents distally with respect to the tubular object’s wall (FIG. 31b). As the peristaltic wave propagates distally over successive segments of the tubular object, the luminal contents are moved along in the proximal-to-distal direction (FIG. 31c).

A stent placed in a biological tubular object capable of peristalsis is inherently vulnerable to migration. However, the scaffold 1 is resistant to migration via peristalsis. When the peristaltic contraction is proximal to the deployed scaffold 1. the most proximal segment of the scaffold 1 is longitudinally compressed and expands radially as a result, effectively forming a “flared” end (FIG. 32b). The wall 60 of the tubular object is pulled over the proximal end of the scaffold 1. When the peristaltic contraction is over the most proximal segment of the scaffold 1, the segment is radially compressed, and concomitantly longitudinally extends (FIG. 32c). The proximal end of the scaffold 1 is pushed proximally, past the section of the tubular object 60′s wall previously slipped over it, back to its original position (i.e. retrograde distal-to-proximal movement). Stretching of the tubular object 60′s wall distal to the peristaltic wave is accommodated by the scaffold 1′s longitudinal extension. The middle segment of the scaffold 1 may be longitudinally compressed by the extending proximal segment, expanding its transverse diameter and increasing its radial grip on the wall 60. When the peristaltic contraction is over the middle segment of the scaffold 1, similar processes occur (FIG. 32d): (1) the proximal segment of the scaffold 1 is pushed proximally past the tubular object 60′s wall and restored to its original position; (2) the middle segment of the scaffold 1 decreases in diameter and increases in longitudinal span to accommodate the stretched overlying wall; and (3) the distal segment may be longitudinally compressed and radially expanded to anchor the scaffold 1 more firmly in place. The processes are repeated (FIG. 32e) until the peristaltic wave passes over the scaffold 1, which remains in the same position with respect to the tubular object 60 (FIG. 32f). Compared to other position fixation mechanisms (flared ends, anchor screws) for stents, the dynamic shape transformation by the scaffold 1 may be less traumatic but more effective against peristalsis-driven migration.

When the scaffold 1 is applied around an object, the radial grip by a helical spline 3a, 3b or receiving groove 4a, 4b of the scaffold 1 is distributed evenly over a longitudinal distance equal to its pitch along the object, so that no section of the object will face a concentrated or circumferential grip (FIGS. 10 - 13) unless the pitch is zero (i.e. the helical spline or receiving groove is a circular ring, which may be the case of an auxiliary wire spline-receiving groove pair, FIGS. 14 and 15). Such a geometric arrangement will prevent crushing of the object by the helical spline 3a, 3b or receiving groove 4a, 4b, or abrasion of the external surface of the object by the helical spline 3a, 3b or receiving groove 4a, 4b during repetitive flexing and unflexing of the assembly. The oppositely facing receiving grooves 4a, 4b locking the principal splines 3a, 3b in place ensure the scaffold 1 is unlikely to be dislodged from repetitive flexing and unflexing of the object and can only be removed by intentional unwinding from its outermost turn.

Unlike traditional stents with complete rings of struts, the scaffold 1 does not place circumferential radial stress on any segment of the wall of the tubular object 60 into which it is placed. Flat strip auxiliary splines 20 at the two ends of the scaffold 1 are intended to be malleable during scaffold deployment and not to exert any radial stress on the tubular object’s wall 60. The absence of circumferential radial stress should reduce or even prevent dissection or thickening of the tubular object’s wall 60 at the edges of the stent.

The struts of a stent may break over time due to fatigue fracture from repetitive flexing and unflexing. The sharp ragged ends of the fracture may perforate the wall of the blood vessel housing the stent. For the scaffold 1 of the present invention, even if the metallic or other rigid polymer components in the principal 3a, 3b or auxiliary 9 splines and/ or receiving grooves 4a, 4b do snap, the sharp ragged ends can be contained in the scaffold membrane 2 if it is made of a material with high tear strength (e.g. orthogonally laminated ePTFE layers).

Expanded PTFE has high tensile strength parallel to its polymer strands, is chemically very inert and will not significantly disintegrate inside the human or animal body. A deployed scaffold 1 made from a membrane 2 containing appropriately arranged ePTFE layers is likely to be retrieved (“explanted”) successfully by pulling without any fragments falling off (and causing embolism), even if the principal splines 3a, 3b and/ or receiving grooves 4a, 4b have fractured at places.

Because of the inherent helical shape of the deployed scaffold 1, it may induce spiral laminar flow within a blood vessel, resulting in physiologically advantageous fluid dynamics, especially at bifurcation sites (blood vessel branch points), and reduction of platelet adhesion (and hence thrombosis).

Referring now to FIG. 26, the scaffold 1101 can be externally applied to a “lead” (an insulated electric cord containing conductor cables) connecting a cardiac implantable electronic device (CIED) or neuro-stimulator to excitable biological tissues (the heart, a nerve, the brain, the spinal cord) in clinical medicine, to:
1. hold the lead securely in position without slipping (so that the lead’s tip will not dislodge from its deployment site);
2. fix the lead’s position (anchor the lead) to an adjacent structure (FIG. 26);
3. protect the lead from conductor fracture due to excessive radial stress applied through sutures (by non-circumferential longitudinally distributed radial grip)
4. provide an extra layer of insulation for the lead body segment within;
5. protect the lead body segment within against tissue ingrowth, inflammation, bacterial colonisation and thrombosis.

A scaffold (with or without an external handle for anchorage to an adjacent anatomical structure) can be applied from the side to a lead which has developed a breach in its external insulation, and will securely attach to the lead even without adhesive. (The current commercially available lead insulation repair kit requires sliding short lengths of silicone tubes over the lead body and fixing them in place with medical adhesives. The short silicone tube is generally oversized with respect to the lead body to be repaired as it has to slide over the connector pin, which is larger in calibre than the body of most leads. Medical adhesives take time to cure and generally do not give very strong bonds.)

A self-extracting lead sleeve can be externally applied to the entire length of a transvenous lead connecting a cardiac implantable electronic device (CIED) or neuro-stimulator to excitable biological tissues (the heart, a nerve, the brain, the spinal cord) to:
1. protect the lead from conductor fracture by reducing the curvature of any bend through distributing it over a longer length:
2. protect the lead against outside-in abrasion;
3. contain any cables that may protrude out of the lead body from inside-out abrasion;
4. protect the lead body against tissue ingrowth, inflammation, bacterial colonisation and thrombosis:
5. make the lead safe and easy to remove (“extract”) even after a long dwell time inside the human or animal body.

Once a transvenous lead has been implanted inside the human or animal body, it can become heavily encased in fibrous tissues and become difficult or even dangerous to extract.

The self-extracting lead sleeve is a scaffold 1401 as shown in FIG. 33 with two nitinol principal splines 1403a, 1403b and a nitinol auxiliary spline 1409 (compulsory auxiliary spline at the distal or lead tip end; optional auxiliary spline at the proximal or lead connector pin end). The splines 1403a, 1403b are in direct physical contact and form a continuous electric circuit. The scaffold 1401 is tightly wrapped around the lead body 74 in the proximal-internal-to-distal topology in a malleable state. The receiving grooves 1404a, 1404b allow radial expansion with preserved longitudinal span through unwinding. When the lead 74 is in service inside the human or animal body, the scaffold 1401 protects the lead 74 from conductor fracture and insulation breach, and contains any insulation breach which may have occurred.

During lead extraction, the lead 74 is exposed at the surgical access site (usually just deep to the subcutaneous tissues in the shoulder or loin region, FIG. 33a). An electric current is passed between the proximal ends of the 2 principal splines 1403a, 1403b linked by the distal auxiliary spline 1409 (FIG. 33b). The principal 1403a, 1403b and the distal auxiliary splines 1409 assume their pre-set shape under ohmic heating. The scaffold 1401 radially expands without longitudinal shortening through unwinding (FIG. 33c). The electric current provides the energy to overcome any resistance from the surrounding tissues against radial expansion of the scaffold 1401. The heat generated by the electric current may also expand the lead tract by shrinking the surrounding tissues through desiccation.

A locking stylet or lead locking device 79 is inserted inside the lumen of the lead 74 to provide tensile strength and distal lead tip control. A sheath 73 is inserted around the lead 74 through the channel newly created within the radially expanded scaffold 1401 all the way to near the lead tip (FIG. 33d). The locking stylet/ lead locking device 79 is used to pull on the lead tip and the sheath 73 is used to provide counter-traction around. Once the lead 74 has been freed at the tip, it is removed with the sheath 73 and replaced with a guide wire 72 (FIG. 33e). The scaffold 1401 is then removed by pulling on its distal end around the guide wire 72 (FIG. 33f). With the proximal-internal-to-distal topology, the scaffold 1401 should disassemble safely and easily tum by turn. The scaffold 1401′s transverse diameter decreases as it is longitudinally extended, and its movement should not be impeded by the surrounding tissues The guide wire 72 forces the scaffold 1401 strip to move into the lumen of the lead tract and stops it from cutting or abrading its external wall even around the inner curvature of an anatomical bend. In this manner, the lead 74 becomes self-extracting, i.e. it carries the means for its own removal at the time of implantation.

Referring now to FIG. 34 there is shown a further embodiment of a scaffold referred to generally by reference numeral 1501. The scaffold comprising a plug 1590 for the pin of a transvenous lead 91. The scaffold 1501 will:
1. seal off the lumen of a transvenous lead from ingress of fluids such as blood as it is transported through a liquid-filled environment such as the central venous or arterial system in the human or animal body;
2. has a low “profile” (small transverse diameter) mean of attaching securely (i.e. will not come loose) to the lead body when transported through a liquid-filled environment such as the central venous or arterial system in the human or animal body;
3. contains a means by which the lead pin can be manipulated remotely through minimal access by grasping tools such as snares, catheters, forceps.

The pin plug 1590 consists of a central cylindrical core 1592 within a cylindrical shell 1593 mounted on a circular end plate 1594 (FIG. 34). The pin plug 1590 will fit into and around the lumen of the connector pin 95 for a lead 91 and seal it off from ingress of fluids such as blood. The scaffold 1501 is joined to the base of the pin plug 1590 and has a pre-set transverse diameter that will grip the lead body 91 strongly when it is externally wrapped around it. The pin plug 1590 has a handle 1596 at its other end. In one simple form, the handle 1596 is a flat circular disc with a diameter the same as the lead body mounted on a cylindrical stalk.

Suppose it is anatomically more convenient, feasible, effective and safer to fix the tip of a transvenous lead to a target site from the groin (the “femoral” approach). After the lead’s tip position has been fixed, the connector pin of the lead needs to be transported from the femoral region, through the bloodstream, and out of the shoulder (“pectoral”) region when the pulse generator of the cardiac implantable electronic device (CIED) will be placed.

The scaffold 1501 which can be referred to as a lead pin plug-handle is externally applied to the lead pin 95 in the femoral region outside the human or animal body. The handle 1596 is then mounted on a “ramrod” dilator 97 with a hemi-spherical tip cut with a planar cleft that will fit snuggly around it (FIG. 35a). The ramrod dilator 97 then delivers the lead connector pin 95 to just beyond the sheath 98a through which the lead 91 has been implanted (FIG. 35b). The sheath 98a has been pre-mounted with a loop snare 99. The snare 99, housed within another sheath 98b, is pulled up to grip the handle 1596 on its stalk (FIG. 35c). The handle 1596 is then pulled or swung out of the ramrod dilator 97 by pulling back the snare 99 further (FIG. 35d). The lead’s connector end is turned through 180 degrees into the sheath 98b housing the snare 99 (FIG. 35e). The snare 99 occupies the space on either side of the circular disc handle 1596 and the overall diameter of the apparatus remains that of the lead’s connector piece. The lead pin 95 and the connector piece are pulled further into the sheath 98b with the snare 99 and the whole assembly is pulled out of the shoulder region to exteriorise the lead pin 95 (FIG. 35f). The lead pin plug-handle and the associated ramrod dilator 97 are dedicated tools that will enable the Jurdham technique to be implemented more safely, effectively through less invasive access.

The scaffold 201 shown in FIG. 6 may be used as a conical stent. A conical stent can be useful in two clinical situations: in a tapering artery or in the slanted “ostium” (origin) of a blood vessel.

For a stenosis in a tapering artery 50 (FIG. 36a), a stent needs to adjust to the smaller diameter at the distal end and the larger diameter at the proximal end of the artery. FIG. 36b shows a stent 51 as known in the art when applied to a tapering artery 50. The distal end of the stent tends to be over-expanded with respect to the artery, whose wall may tear and bleed (edge dissection or intra-mural haematoma). The arterial segment distal to the stent may go into spasm in response to the mechanical injury, resulting in a vessel calibre smaller than before stenting. The proximal end of the stent tends to be under-expanded with respect to the artery, whose wall may not be completely apposed by the stent struts. Stent mal-apposition may lead to thrombosis. A conical stent, such as scaffold 201, intrinsically has a transverse diameter that varies along its longitudinal span, allowing the central stenosis to be adequately supported without distal arterial wall damage and proximal stent mal-apposition (FIG. 36c).

A slanted ostium cannot be perfectly covered by a cylindrical stent for geometrical reasons: either the stent leaves a short segment of the side branch uncovered (provisional T stenting) or a short segment of the stent protrudes into the lumen (T stenting and small protrusion). A conical stent. e.g. scaffold 201, can be used in a novel way to overcome this geometric conundrum in the following manner:
1. A conical stent 201 is mounted on an inflatable balloon 75 and positioned partially in and partially out of the slanted ostium 53 via a guide wire 59a (FIG. 37a).
2. The balloon 56a is inflated and the conical stent 201 assumes its expanded state. With longitudinal contraction concomitant with radial expansion, the proximal end of the conical stent 201 becomes flush with or distal to the short edge of the slanted ostium 53 (FIG. 37b).
3. The deployment balloon 56a is exchanged for a shorter inflatable balloon 56b positioned to cover just the proximal half of the deployed conical stent 201 (FIG. 37c).
4. The shorter balloon 56b is inflated to engage the proximal but not the distal half of the deployed conical stent 201 (FIG. 37d).
5. The shorter balloon 56b is pulled back proximally, dragging the proximal half of the conical stent 201 out with it past the slant ostium, dislodging and deforming the stent 201 at the same time (i.e. intentional controlled longitudinal stent deformation, FIG. 37e).

The proximal end of the conical stent 201 is then tilted so that it becomes flush with the slant ostium. The technique differs depending on whether a wall opposing the slanted ostium is available in practice.

If an opposing wall is available:
1. The shorter balloon 56b is deflated and advanced into the distal half of the conical stent 201 (FIG. 38a). A second longer and larger balloon 56c is positioned over a second guide wire 59b between the slanted ostium 53 and its opposing wall 57.
2. The second longer balloon 56c and the shorter balloon 56b are inflated simultaneously (FIG. 38b). The shorter balloon 56b anchors the distal half of the conical stent 201 and prevents it from being dislodging more distally. The larger balloon 56c deforms the proximal section of the conical stent 201 to make its end flush with the slanted ostium 53.
3. Both balloons 56b, 56c are deflated and removed, leaving the proximal end of the conical stent 201 flush with the slanted ostium 53 (FIG. 38c).

If an opposing wall is unavailable:
1. A larger and longer balloon 56c is advanced over the stiff portion of a second angioplasty guide wire 59b to become partly inside and partly outside the guide catheter 58 positioned at the slanted ostium 53 (FIG. 39a).
2. The larger 56c and the smaller 56b balloons are inflated simultaneously (FIG. 39b). The smaller “anchor” balloon 56b is pulled while the guide catheter 58 is advanced simultaneously. The stiff portion of the guide wire 59b, the inflated larger balloon 56c and the guide catheter 58 under tension together form a system rigid enough to flatten the protruding segment of the conical stent 201 against the slanted ostium 53.
3. Both balloons 58b, 56c, both guide wires 59a, 59b and the guide catheter 58 are withdrawn, leaving the proximal end of the conical stent 201 flush with the slanted ostium 53 (FIG. 39c).

A conical stent made out of the scaffold is especially advantageous for the technique described for several reasons.

1. With the proximal-internal-to-distal topology, the proximal turns of the deployed stent can be pulled back more proximally and advanced more distally relatively easily.
2. With multiple receiving grooves, the principal splines dislodged from their positions in the ideal pre-set cone shape may still become locked in other receiving grooves in the sheared deformed stent.
3. The conical scaffold naturally has wider membrane overlap towards its broad end, and so adequate cover of the slanted ostium can still be maintained even if the scaffold is stretched over a larger area.
4. As the conical stent is deployed in an extended state, it will try to recoil into a shorter longitudinal span, shifting any excess stent materials from outside to inside the slanted ostium and increasing the radial expansion force against the ostial wall.

A scaffold can form an ePTFE covered stent for the coronary, peripheral, carotid and vertebral arteries, and the aorta. If the ePTFE is infused with a perfluorocarbon, the scaffold will have a SLIPS. A SLIPS stent will resist, reduce or prevent:
1 thrombosis, by reducing or preventing:
   a. platelet aggregation (without need of orally taken anti-platelet drugs);
   b. mal-apposition against the artenal wall;
   c. infolding of stent wall;
   d. protrusion of fractured stent struts (will be contained within the ePTFE scaffold membrane which has excellent tensile strength and biochemical stability).
2. restenosis by neo-intimal hyperplasia without drug elution, by;
   a. having no gaps in its wall;
   b. having a non-inflammatory SLIPS;
3. edge stenosis, by having no circumferential radial stress along the entire longitudinal span of the stent, including the edges;
4. dislodgement (counteracted by longitudinal compression mediated radial expansion);
5. embolisation of debris from the arterial wall during stenting by trapping it under its covered wall;
6. embolisation of polymer coating fragments:
7. covering or colonisation by endothelial cells, smooth muscle cells, fibroblast and bacteria.
8. kinking due to the telescopic helix formation inherent in the stent’s structure (especially important for use in the peripheral arteries).

A SLIPS stent should remain pristine long after implantation because of the biochemical inertness of perfluorocarbons. This will allow the stent to be retrieved using the auxiliary splines as internal handles on the stent (FIGS. 23 and 25). The laminating layer for the spanning membranes can be made to contain windows to allow their fenestration (perforation by a stiff guide wire followed by dilatation by an inflatable balloon) in clinical use. This will allow side branch access from the covered stent. The stent may also induce spiral laminar blood flow.

The scaffold can form a vascular graft that can be rapidly externally applied to a leaking blood vessel (e.g. a ruptured aortic aneurysm). As the scaffold is self-assembling, the user only needs to wrap the scaffold strip roughly around the leaking blood vessel and the splines will slot into the receiving grooves semi-automatically. This is important as the leak in the blood vessel can often not be clearly visualised because of the amount of blood gushing out. Once the immediate blood loss has been staunched, the damaged blood vessel can be fixed permanently. The scaffold can be incorporated as part of the permanent surgical repair. If the scaffold is infused with perfluorocarbons to form a SLIPS, the resulting vascular graft may be resistant to thrombosis, infection and stenosis. Because of the biochemical inertness of ePTFE (with or without infusion with perfluorocarbons), the graft will probably never be endothelialised and incorporated into the body.

A SLIPS scaffold can be made into a flexible, kink resistant long term indwelling catheter (e.g. for haemodialysis, chemotherapy, urinary tract), a prosthetic vascular graft (e.g. between an artery and a vein in the formation of arterio-venous fistula for haemodialysis; between the aorta and the coronary arteries in coronary artery bypass surgery; for the carotid artery. or between the femoral and popliteal arteries) or plasticiser free flexible tubing (which can be used for liquid infusion in clinical practice) by putting in extra pairs of receiving grooves (to absorb compression and extension around bends and induce spiral laminar flow within) and sealing the spline-groove joints with a liquid proof adhesive (FIGS. 7 and 8). SLIPS will protect the catheter or graft against thrombosis, encrustation with debris, colonisation by bacteria (and hence infection) and body cells (and hence catheter or graft stenosis by tissue ingrowth).

The scaffold can be used to form a stent for tubular organs capable of peristalsis and carrying luminal flow (e.g. the bile duct, the oesophagus, the colon, the stomach, the ureters; FIGS. 31 and 32). The tubular structures may be subjected to external compression by tumour growth or fibromuscular overgrowth. Stent loss may also be caused by encrustation of the luminal contents on the stent causing obstruction, tumour ingrowth through gaps between the stent struts (uncovered stents) and at the ends of the stent, migration, and trauma to the tubular organs (dissection, haemorrhage). Stent migration is a major issue as organs capable of peristalsis are evolutionarily designed to expel their contents. The scaffold intrinsic coil spring like mechanical properties may be able to resist peristalsis. The scaffold is also a covered stent with no circumferential radial stress, even at the edges. The scaffold can be deployed or retrieved with minimal body invasion (FIGS. 23 - 25).

The scaffold can be wrapped externally around soft tubular organs of the pelvic floor (e.g. the vagina, the urethra, the rectum) that are prone to prolapse (with ageing, weakening of the pelvic floor from childbirth, previous surgery, previous radiation therapy) to provide flexible mechanical support. The scaffold can be pre-set to have an internal diameter that will not impede the flow of the contents of these soft tubular organs. The scaffold has intrinsic longitudinal and radial elasticity and should not feel rigid for the recipient. If the scaffold membrane is made of ePTFE (with or without infusion of perfluorocarbons), the scaffold should resist ingrowth by the surrounding tissues, making the scaffold easy to remove surgically if that proves necessary later.

The scaffold can be wrapped around an electric cable to:
1. provide electric insulation if the cable’s own insulation has breached;
2. protect an electric cable from breaking (insulation breach or conductor fracture) through reduction of curvature, especially at the junction between a relatively rigid section and a relatively flexible section of the cable.

The scaffold can be externally applied to an electric cable from the side even if both of its ends are attached to significantly larger objects (e.g. an integrated plug) without any other apparatus (e.g. a heat gun for heat-shrink tubing). Unlike other electric cable insulation repair kits, the scaffold will attach securely to an electric cable but can be easily dismantled from one electric cable and reused on another. A single scaffold can also be wrapped around multiple electric cables to organise them into a manageable bundle, and provide the means by which the cables can be tied down through one or more anchorable handles (FIG. 26).

The scaffold can be wrapped around and then pulled tight around a leakage pipe. The splice-groove joints can be equipped with a liquid proof adhesive to produce a leak proof seal. The ends of the scaffold may be compressed with another pair of externally applied clamps to contain the hydraulic pressure. The scaffold can be left as a temporary, semi-permanent or permanent fix to the leak. The scaffold and deployment/ retrieval techniques from remote minimal access can be adapted in other internal liquid or gas pipe repair jobs.

Referring now to FIGS. 40 and 41 there is shown a further embodiment of a scaffold according to the invention, referred to generally by reference numeral 1601. The scaffold 1601 is adapted to allow the easy manufacture of flexible, compact (tight packing of electrode materials and hence high energy density), cylindrical batteries. The scaffold 1601 only has a pair of receiving grooves 1604a, 1604b right next to the principal splines 1603a, 1603b (FIG. 40). The panel of scaffold membrane 1602 spanning between the receiving grooves 1604a, 1604b has multiple layers. Starting from the external surface towards the internal (luminal) surface of the rolled telescopic cylindrical helix formation, the membrane 1602 comprises one or more layers of ePTFE 1618a. a laminating layer of FEP 1619 (impermeable), a thin (cathode) current conductor strip 1628 (e.g. made out of aluminium foil), the cathode 1626 (e.g. carbon monofluoride, manganese dioxide, generally mixed with other binding materials into a paste), and one of more layers of ePTFE 1618b (semi-permeable). The scaffold 1601 can be wrapped around a central anode core 1632 (e.g. lithium metal) containing a central (anode) current collector 1629 which is also malleable/ flexible (e.g. a copper wire, a silver wire, an aluminium wire, a graphene string, a carbon nanotube construct) (FIG. 41). The spline-groove joints are sealed with an elastic but impermeable adhesive. Lithium is highly malleable and can be easily be shaped with grooves or indentations to accommodate the bulges of the cathode paste. The semi-permeable luminal ePTFE layers 1618b allow the passage of ions (electrolytes) and solvents and can be made to be extremely thin to minimise the internal resistance of the battery. The luminal layers can also be made to be extremely strong against tear (e.g. by orienting successive layers of ePTFE so that their fibrils lie orthogonally) to prevent the cathode 1626 and the anode 1632 coming into direct physical contact (which would generate an internal short circuit of the battery and a runaway electrochemical and thermal reaction). The FEP laminating layer 1619 seals up the entire battery (except for connections for the current collectors) and prevents the leakage of its contents (mainly the solvents). The external layer 1618a can be impregnated with a perfluorocarbon to make the entire battery resistant against tissue ingrowth, thrombosis (blood clot formation) and bacterial colonisation. Such a flexible cylindrical high energy density will be very useful for powering CIEDS (e.g. a leadless pacemaker, an implantable “string” subcutaneous defibrillator). However, the same battery will also be useful for powering other non-medical consumer electronic products.

In one embodiment, the scaffold may be referred to as a Self-Assembling Extendible Expandable Retrievable scaffold (i.e. SAFEER scaffold) that can be applied to and removed from a tubular object either on the external surface from the outside, or on the internal surface through an interior channel (the “lumen”); whether the tubular object is rigid or flexible, static or subjected to repetitive deformation; with relatively ease and minimal training of the operator; even when direct physical access to the object is restricted. The SAFEER scaffold can be used to provide mechanical support to the structural integrity of the object, which is flush with its ends (even if they are slanted with respect to its longitudinal axis), conforms to the object’s wall even if its cross-section profile, transverse diameters and curvature vary along its length, protect the tubular object and its contents from damage caused by repetitive flexing and unflexing (i.e. fatigue fracture), external or internal abrasion, or any other forms of physical and chemical insults. The SAFEER scaffold can be used to form a continuous surface lining the lumen or covering the external surface of the object that stops, prevents or reduces: leakage across the object’s wall out of or into the lumen; thrombus (blood clot) formation (if the object is a blood vessel); adhesion by biological entities (cells and micro-organisms) and their secretions, or other organic or inorganic particles. The SAFEER scaffold can produce a physical gap on demand (which may require the application of a stimulus or an energy source) separating the object from its surroundings (even against constricting and restricting influences), so that: another instrument can be inserted alongside or over the tubular object within the same surroundings; the object can be moved freely with respect to (and hence removed safely from) its surroundings. The SAFEER scaffold can be adapted to have one or more handles that can be used to: anchor the scaffold (and indirectly the tubular object) to the surroundings; provide purchase for a manipulation or retrieval tool, directly for the scaffold, or indirectly for the tubular object through the scaffold.

In relation to the detailed description of the different embodiments of the invention, it will be understood that one or more technical features of one embodiment can be used in combination with one or more technical features of any other embodiment where the transferred use of the one or more technical features would be immediately apparent to a person of ordinary skill in the art to carry out a similar function in a similar way on the other embodiment.

In the preceding discussion of the invention, unless stated to the contrary, the disclosure of alternative values for the upper or lower limit of the permitted range of a parameter, coupled with an indication that one of the values is more highly preferred than the other, is to be construed as an implied statement that each intermediate value of the parameter, lying between the more preferred and the less preferred of the alternatives, is itself preferred to the less preferred value and also to each value lying between the less preferred value and the intermediate value.

The features disclosed in the foregoing description or the following drawings, expressed in their specific forms or in terms of a means for performing a disclosed function, or a method or a process of attaining the disclosed result, as appropriate, may separately, or in any combination of such features be utilised for realising the invention in diverse forms thereof.

1. A scaffold for a tube, the scaffold comprising a membrane and a pair of splines integrally formed with or embedded in the membrane, the splines being spaced apart from one another with the membrane spanning therebetween, the membrane further comprising a pair of grooves disposed between the splines adapted to receive the splines when the membrane is folded over on itself, wherein one groove is engaged with one spline and the other groove engages with the other spline.

2. A scaffold as claimed in claim 1 wherein the scaffold has a flattened configuration and a rolled configuration and wherein the scaffold is transformable between the flattened and rolled configurations.

3. A scaffold as claimed in claim 2 wherein, in the rolled configuration the splines and grooves are helical in shape.

4. A scaffold as claimed in claim 3 wherein the scaffold is substantially cylindrical in the rolled configuration.

5. A scaffold as claimed in claim 3 wherein the scaffold is substantially conical in the rolled configuration.

6. A scaffold as claimed in claim 5 wherein the cone has a narrow diameter end near the apex and a wide diameter end at the base.

7. A scaffold as claimed in claim 6 wherein the grooves and splines diverge from one another in a direction from the narrow diameter end towards the wide diameter end.

8. A scaffold as claimed in any preceding claim wherein the scaffold is telescopic in the rolled configuration such that it can longitudinally expand or retract.

9. A scaffold as claimed in any preceding claim wherein the splines and/or the receiving grooves are formed of shape memory materials that will assume a pre-set spiral shape at predetermined temperature such that the scaffold self assembles into the rolled configuration at a predetermined temperature.

10. A scaffold as claimed in any preceding claim wherein the pair of splines are principal splines and the scaffold comprises one or more auxiliary splines, the auxiliary splines being disposed proximal to one or both longitudinal ends of the scaffold.

11. A scaffold as claimed in any preceding claim wherein the scaffold comprises one or more handles to facilitate deployment and retrieval of the scaffold.

12. A scaffold as claimed in claim 11 when dependent on claim 10 wherein the one or more handles are formed from auxiliary splines.

13. A scaffold as claimed in claim 11 or claim 12 wherein the one or more handles are formed from a material having shape memory such as nitinol.

14. A scaffold as claimed in claim 13 wherein the one or more handles may be folded away from the longitudinal axis of the scaffold in the rolled configuration, and wherein upon reaching a pre-set temperature, the one or more handles fold towards the longitudinal axis of the scaffold in the rolled configuration.

15. A scaffold as claimed in any one of claims 11 to 14 wherein the handle is anchorable to a surface.

16. A scaffold as claimed in claim 15 wherein the handle is anchorable to a surface via sutures.

17. A scaffold as claimed in any preceding claim wherein the scaffold membrane is impregnated with a lubricant such as perfluorocarbons.

18. A scaffold as claimed in any preceding claim wherein the scaffold membrane is formed from two or more membrane layers.

19. A scaffold as claimed in any preceding claim wherein the scaffold membrane comprises expanded polytetrafluoroethylene.

20. A scaffold as claimed in any preceding claim wherein the scaffold membrane comprises fluorinated ethylene propylene.

21. A scaffold as claimed in any preceding claim wherein the scaffold is engineered to release molecules into the surrounding environment.

22. A scaffold as claimed in any preceding claim wherein the scaffold has a plurality of rolled configurations, wherein different rolled configurations provide different diameters.

23. A scaffold as claimed in any preceding claim wherein the scaffold comprises a plurality of pairs of grooves.

24. A scaffold as claimed in any preceding claim wherein the scaffold is fixable to a pin plug for connecting the scaffold to the connector pin of a lead used with a CIED or a neuro-stimulator.

25. A scaffold as claimed in any preceding claim wherein the scaffold is adapted for use in the manufacture of batteries and wherein the scaffold membrane comprises a plurality of layers, the scaffold membrane comprising a current conductor strip and a cathode and wherein the current conductor strip and the cathode are sandwiched between structural layers.

26. A scaffold as claimed in any preceding claim wherein when the scaffold in the rolled configuration is bent along its longitudinal axis, the overlap between turns of the scaffold ensures a continuous surface is maintained, even if part of the splines is no longer locked in the receiving grooves.

27. A method for retrieving the scaffold as claimed in claim 14 from a tube, the method comprising the step of inserting an inflatable balloon into the lumen of the scaffold and inserting a heated substance into the balloon to inflate the balloon and heat the scaffold such that the shape of the scaffold is altered by the heat thereby trapping the balloon in the scaffold, then drawing the balloon and the scaffold out of the tube.

28. A battery comprising a scaffold for a tube as claimed in claim 25.

29. A method of manufacturing a battery as claimed in claim 28, the method comprising the steps of providing a central anode core containing a flexible central current collector, and wrapping the central anode core with the scaffold, the scaffold comprising a current conductor strip and a cathode.
